(19) United States
(12) Patent Application Publication
(10) Pub. No.: US 2018/0153995 A1
(43) Pub. Date: Jun. 7, 2018

(54) USE OF ANTIMICROBIAL POLYMERS FOR RE-SENSITIZATION OF MICROORGANISMS UPON EMERGENCE OF RESISTANCE TO ANTI-MICROBIAL AGENTS

(71) Applicant: Technion Research & Development Foundation Limited, Haifa (IL)

(72) Inventors: Amram Mor, Nesher (IL); Fadia Zaknoon, Haifa (IL); Shahar Rotem, Kiryat-Tivon (IL); Hadar Sarig, Kfar-Yona (IL)

(73) Assignee: Technion Research & Development Foundation Limited, Haifa (IL)

(21) Appl. No.: 15/844,557

(22) Filed: Dec. 17, 2017

Related U.S. Application Data
(63) Continuation of application No. 12/812,965, filed on Jul. 15, 2010, now abandoned.
(60) Provisional application No. 61/021,474, filed on Jan. 16, 2008.

(51) Int. Cl. A61K 45/06; A61K 38/10; A61K 31/785; A61K 31/7048; A61K 31/7036; A61K 38/08; A61K 31/496; A61K 31/431; A61K 31/43; A61K 31/56

(52) U.S. Cl. CPC A61K 45/06; A61K 38/10; A61K 31/785; A61K 31/7048; A61K 31/56; A61K 38/08; A61K 31/496; A61K 31/431; A61K 31/43; A61K 31/7036

(57) ABSTRACT

Methods and compositions for treating microbial infections associated with an emergence of resistance of a pathogenic microorganism to an antimicrobial agent, following treatment with antimicrobial agent are disclosed. The methods are effected by using a polymer which exhibits antimicrobial re-sensitizing activity, for re-sensitizing the pathogenic microorganisms to the antimicrobial agent, in combination with the antimicrobial agent. Further disclosed are novel polymers having an antimicrobial re-sensitizing activity.

### RELATED APPLICATIONS

This Application is a continuation of U.S. application Ser. No. 12/812,965 filed on Jul. 15, 2010, which is a National Phase of PCT Patent Application No. PCT/IL2009/000063 having International Filing Date of Jan. 15, 2009, which claims the benefit of U.S. Provisional Patent Application No. 61/021,474 filed on Jan. 16, 2008. The contents of the above Applications are all incorporated herein by reference.

### SEQUENCE LISTING STATEMENT

The ASCII file, entitled 71346SequenceListing.txt, created on Dec. 17, 2017 comprising 6,257 bytes, submitted concurrently with the filing of this application is incorporated herein by reference.

### FIELD AND BACKGROUND OF THE INVENTION

The present invention, in some embodiments thereof, relates to medicinal treatments directed at overcoming an emergence of resistance to antimicrobial treatment, and more particularly, to use of a class of polymers which exhibit a re-sensitizing effect against antimicrobial-resistance developed in subjects having a microbial infection, following an antimicrobial treatment.

Antibiotics, which are also referred to herein and in the art as antibacterial or antimicrobial agents, constitute one of the greatest triumphs of modern medical science, ever since their discovery and recognition by Alexander Fleming in 1928.

Natural and synthetic antimicrobial agents have been developed and used for decades with great success and virtually transformed the survival rates of infected subjects all over the world. However, over the decades, almost all the prominent infection-causing bacterial strains have developed resistance to currently available antibiotics.

The emergence of antimicrobial-resistance is an evolutionary process that is based on natural and induced selection for microorganisms that acquired the ability to proliferate to some extent in the presence of antimicrobial agents, which previously were able to eradicate these microorganisms. Example for this phenomenon is seen in the antibiotics penicillin and erythromycin, which were considered miracle drugs but are now far less effective due to the fact that bacteria have become more resistant thereto. Resistance is often inheritable and in most cases caused by excessive use of antibiotics, which themselves exert a selective pressure which allows the growth of resistant bacteria within a population and inhibits susceptible bacteria. Molecular mechanisms leading to antimicrobial resistance include intrinsic resistance which may occur naturally as a result of the microorganism's genetic makeup, wherein the microbial chromosome may fail to encode a protein which the antimicrobial agent targets. Another mechanism includes acquired resistance, which results from a mutation in the microbial chromosome or the acquisition of extra-chromosomal DNA. The spread of antimicrobial resistance between different microorganisms may also be mediated by horizontal transfer of plasmids that carry genes which encode antimicrobial resistance, and may result in co-resistance to multiple antibiotics.

Antibiotic resistance can result in severe adverse outcomes, such as increased mortality, morbidity and medical care costs for patients suffering from common infections, once easily treatable with antibiotics (Am. J. Infect. Control 24 (1996), 380-388; Am. J. Infect. Control 27 (1999), 520-532; Acar, J. F. (1997), Clin. Infect. Dis. 24, Suppl 1, S17-S18; Cohen, M. L. (1992), Science 257, 1050-1055; Cosgrove, S. E. and Carmeli, Y. (2003), Clin. Infect. Dis. 36, 1433-1437; Holmberg, S. D. et al. (1987), Rev. Infect. Dis. 9, 1065-1078) and therefore became one of the most recognized clinical problems of today's governmental, medicinal and pharmaceutical research (U.S. Congress, Office of Technology Assessment, Impacts of Antibiotic-Resistant Bacteria, OTA-H-629, Washington, DC, U.S. Government Printing Office (1995); House of Lords, Science and Technology 7th Report: Resistance to Antibiotics and Other Antimicrobial Agents, HL Paper 81-II, session (1997-98); and Interagency Task Force on Antimicrobial Resistance, A Public Health Action Plan to Combat Antimicrobial Resistance. Part 1: Domestic issues).

Due to the limitations associated with the use of classical antibiotics, extensive studies have been focused on finding ways to limit, and overcome, the emergence of microbial resistance towards, antimicrobial/antibacterial agents.

Within these studies, a novel class of short, naturally occurring peptides, which exert outstanding antimicrobial/antibacterial activity, was uncovered. These antimicrobial proteins and peptides (AMPs) constitute a vast family of compounds currently under study which are typically characterized by a flexible structure, an amphiphatic character and a net positive charge. AMPs are typically derived from animal sources and constitute a large and diverse family of peptides. In the past 25 years, over 700 AMPs derived from various sources, from unicellular organisms to mammalians and including humans, have been identified [Gordon, Y. J., E. G. Romanowski, et al. (2005), Curr Eye Res 30(7): 505-15; Stallmann, H. P., C. Faber, et al. (2006), Injury 37 Suppl 2: S34-40; and Yedery, R. D. and K. V. Reddy (2005), Eur J Contracept Reprod Health Care 10(1): 32-42]. Naturally occurring AMPs, and de-novo AMPs having artificially designed sequences, either synthesized by humans or genetically engineered to be expressed in organisms, exhibit various levels of antibacterial and antifungal activity as well as lytic activity toward mammalian cells. As a result, AMPs are attractive targets for bio-mimicry and peptidomimetic development, as reproduction of critical peptide biophysical characteristics in an unnatural, sequence-specific oligomer should presumably be sufficient to endow antibacterial efficacy, while circumventing the limitations associated with peptide pharmaceuticals (Latham, P. W. (1999), Nat. Biotechnol. 17, 755-757).

Peptidomimetic AMPs are modified polypeptides or polymers which are designed to have a superior stability, both in vivo and ex vivo, and yet at least the same receptor affinity, as compared with the peptides they mimic. In order to design efficacious peptidomimetics, a careful attention must be drawn to the characteristics which are responsible for their interaction with the intended target is therefore required.

U.S. Patent Application Nos. 20070032428, 11/234,183 and 11/500,461 and WO 2006/035431, by one of the present inventors, which are incorporated herein by reference as if fully set forth herein, teach a novel class of peptidomimetic antimicrobial polymers. These antimicrobial polymers are composed of hydrophobic moieties and amino acids, and maintain three key attributes of AMPs: a flexible structure, an amphiphatic character and a net positive charge. As presented in these patent applications, these antimicrobial polymers are composed of positively charged amino acid residues, such as lysine, and non-amino acid hydrophobic moieties, such as ω-amino-fatty acid residues, as well as fatty acid residues, which not only achieve the desired amphiphatic trait and resolve the production and maintenance issues limiting the use of polypeptides as drugs, but also alleviate the sever limitations restricting the administration of polypeptides as drugs.

As further demonstrated in U.S. Patent Application Nos. 20070032428, 11/234,183 and 11/500,461 and WO 2006/035431, this newly developed class of polymers has been shown to exhibit high antimicrobial activity, low resistance induction, non-hemolyticity, resistibility to plasma proteases and high affinity to microbial membranes.

Other documents teaching aspects of these biologically active polymers, based on ω-amino-fatty acid residues and positively charged amino acid residues, include WO 2008/072242, teaching compositions and methods for concentrating and depleting microorganisms and WO 2008/132738, teaching anticancerous polymeric agents, which are all incorporated herein by reference as if fully set forth herein.

### SUMMARY OF THE INVENTION

The present invention, in some embodiments thereof, relates to medicinal treatments directed at overcoming a resistance emerged upon antimicrobial treatment, and more particularly, to use of a class of polymers which exhibit a re-sensitizing effect against antimicrobial-resistance emerged in subjects having a microbial infection, following an antimicrobial treatment.

The methods, uses and compositions presented hereinbelow, are directed at treating persistent medical conditions which are caused by pathogenic microorganisms in subjects that were already treated with an antimicrobial agent unsuccessfully, due to the emergence of antimicrobial resistance towards that antimicrobial agent.

Hence, following the unsuccessful treatment with the antimicrobial agent due to the emergence of an antimicrobial resistance, re-sensitization of the pathogenic microorganisms to the antimicrobial agent is achieved by introducing re-sensitizing agents in the form of the polymers described herein, which are administered in combination with the antimicrobial agent.

The antimicrobial re-sensitizing polymers, as described herein, can thus provide valuable therapeutic alternatives, particularly when resistance to antibiotics limits therapeutic options.

The antimicrobial polymers described herein were previously described as exhibiting an antimicrobial activity. Nonetheless, when used as re-sensitizing agents for overcoming an emergence of resistance to an antimicrobial treatment, substantially lower effective amounts of the polymer are required in order to achieve the desired effect.

Thus, according to one aspect of the present invention there is provided a method of treating a medical condition associated with a pathogenic microorganism and further associated with an emergence of antimicrobial resistance in a subject having the medical condition and treated with an antimicrobial agent, the method comprising:

administering to the subject, following a treatment with the antimicrobial agent and the emergence of the antimicrobial resistance, a re-sensitizing effective amount of a polymer which comprises a plurality of positively charged amino acid residues and more than one ω-amino-fatty acid residue, wherein the ω-amino-fatty acid residue is being covalently linked to at least two amino acid residues in the plurality of positively charged amino acid residues via the N-alpha of one amino acid residue and via the C-alpha of the other amino acid residue in the at least two amino acid residues; and administering to the subject a therapeutically effective amount of the antimicrobial agent.

According to some embodiments of the invention, the re-sensitizing effective amount is lower than a therapeutically effective amount of the polymer with respect to the microorganism.

According to some embodiments of the invention, the antimicrobial agent is administered concomitant with or subsequent to administering the polymer.

According to another aspect of the present invention there is provided a use of a polymer as described herein, in the manufacture of a medicament for treating a medical condition associated with a pathogenic microorganism and further associated with an emergence of antimicrobial resistance in a subject having the medical condition and treated with an antimicrobial agent, the medicament being used in combination with the antimicrobial agent and being such that a re-sensitizing effective amount of the polymer is used, the re-sensitizing effective amount being lower than a therapeutically effective amount of the polymer with respect to the pathogenic microorganism.

According to some embodiments of the invention, when the polymer is used in combination with the antimicrobial agent, the antimicrobial agent is administered concomitant with or subsequent to administering the polymer.

According to another aspect of the present invention there is provided a pharmaceutical composition comprising, as active ingredients, a polymer as described herein and an antimicrobial agent, and a pharmaceutically acceptable carrier.

According to some embodiments of the invention, the composition is being packaged in a packaging material and identified in print, in or on the packaging material, for use in the treatment of a medical condition associated with a pathogenic microorganism and further associated with an emergence of antimicrobial resistance in a subject having the medical condition and treated with an antimicrobial agent.

According to another aspect of the present invention there is provided a method of re-sensitizing a pathogenic microorganism to an antimicrobial agent, following a treatment of the pathogenic microorganism with the antimicrobial agent and a subsequent emergence of a resistance of the pathogenic microorganism to the antimicrobial, the method is effected by contacting the pathogenic microorganism with a re-sensitizing effective amount of a polymer as described herein; the re-sensitizing effective amount being lower than a therapeutically effective amount of the polymer with respect to the pathogenic microorganism.

According to some embodiments of the invention, the method includes contacting the microorganism with the polymer comprises administering to a subject having a medical condition associated with the microorganism and further associated with an emergence of antimicrobial resistance in the subject having the medical condition and treated with an antimicrobial agent, the re-sensitizing effective amount of the polymer.

According to some embodiments of the invention, the method further includes administering to the subject the antimicrobial agent.

According to some embodiments of the invention, the antimicrobial agent is administered concomitant with or subsequent to administering the polymer.

According to some embodiments of the invention, the method further includes contacting the pathogenic microorganism with the antimicrobial agent.

According to some embodiments of the invention, contacting the pathogenic microorganism with the antimicrobial agent is effected concomitant with or subsequent to contacting the microorganism with the polymer.

According to another aspect of the present invention there is provided a use of a polymer as described herein, in the manufacture of a medicament for re-sensitizing a pathogenic microorganism to an antimicrobial agent following a treatment of the pathogenic microorganism with the antimicrobial agent and a subsequent emergence of a resistance of the pathogenic microorganism to the antimicrobial, wherein a re-sensitizing effective amount of the polymer is used, the re-sensitizing effective amount being lower than a therapeutically effective amount of the polymer with respect to the pathogenic microorganism.

According to some embodiments of the invention, the polymer is used in combination with the antimicrobial agent.

According to some embodiments of the invention, the antimicrobial agent is administered concomitant with or subsequent to administering the polymer.

According to another aspect of the present invention there is provided a pharmaceutical composition unit dosage form comprising a re-sensitizing effective amount of a polymer as described herein; the re-sensitizing effective amount being such that effects a re-sensitization of a pathogenic microorganism to an antimicrobial agent, following a treatment of the pathogenic microorganism with the antimicrobial agent and a subsequent emergence of a resistance of the pathogenic microorganism to the antimicrobial agent, wherein the re-sensitizing effective amount is lower than a therapeutically effective amount of the polymer with respect to the pathogenic microorganism.

According to another aspect of the present invention there is provided a pharmaceutical kit comprising a packaging material and a polymer as described herein and an anti-microbial agent being individually packaged in the packaging material, the kit being labeled for treating a medical condition associated with a pathogenic microorganism and further associated with an emergence of antimicrobial resistance in a subject having the medical condition and treated with the antimicrobial agent and/or for re-sensitizing a pathogenic microorganism to the antimicrobial agent, following a treatment of the pathogenic microorganism with the antimicrobial agent and a subsequent emergence of a resistance of the pathogenic microorganism to the antimicrobial agent.

According to some embodiments of the invention, in the polymer described herein, the ω-amino-fatty acid is linked to each of the amino acid residues via a peptide bond.

According to some embodiments of the invention, the polymer is a linear polymer or a cyclic polymer.

According to some embodiments of the invention, the plurality of positively charged amino acid residues comprises from 2 to 50 amino acid residues.

According to some embodiments of the invention, the positively charged amino acid residues are selected from the group consisting of lysine residues, histidine residues, ornithine residues, arginine residues and combinations thereof.

According to some embodiments of the invention, the positively charged amino acid residues are lysine residues.

According to some embodiments of the invention, the polymer comprises from 1 to 50 ω-amino-fatty acid residues.

According to some embodiments of the invention, the ω-amino-fatty acid residue is selected from the group consisting of 4-amino-butyric acid residue, 8-amino-caprylic acid residue, 10-amino-decanoic acid residue, 12-amino-lauric acid residue, 14-amino-tetradecanoic acid residue and 16-amino-palmitic acid residue.

According to some embodiments of the invention, the polymer includes more than one fatty acid residue.

According to some embodiments of the invention, the fatty acid residue is selected from the group consisting of butyric acid residue, caprylic acid residue, decanoic acid residue, lauric acid residue, tetradecanoic acid residue, palmitic acid residue, 5-dodecenoic acid residue, dodec-7-enoic acid residue, myristoleic acid residue, tetradec-9-enoic acid residue, tetradec-5-enoic acid residue, hexadec-9-enoic acid residue, and hexadec-7-enoic acid residue.

According to some embodiments of the invention, the polymer has the general Formula I or II:

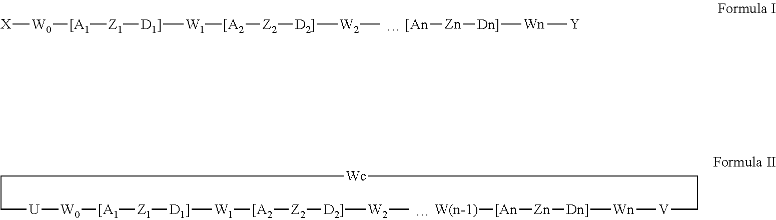

wherein:

n is an integer from 2 to 50;

A₁, A₂, . . . , An are each independently a positively charge amino acid residue;

D₁, D₂, . . . , Dn are each independently an ω-amino-fatty acid residue or absent, provided that more than one of the D₁, D₂, . . . , Dn is the ω-amino-fatty acid residue;

Z₁, Z₂, . . . , Zn and W₀, W₁, W₂, . . . , Wn are each independently a linking moiety linking an amino acid residue and a hydrophobic moiety residue, or absent;

X and Y are each independently selected from the group consisting of hydrogen, amine, amide, a positively charged amino acid residue, an ω-amino-fatty acid residue, a fatty acid residue or absent;

W₀ is a linking moiety linking one of the A₁, Z₁ and D₁ to U, or absent;

Wn is a linking moiety linking one of the An, Zn and Dn to V, or absent;

U is selected from the group consisting of a first functional group, an amino acid residue having the first functional group, a hydrophobic moiety residue having the first functional group, and a linking moiety having the first functional group or absent;

V is selected from the group consisting of a second functional group, an amino acid residue having the second functional group, a hydrophobic moiety residue having the second functional group, and a linking moiety having the second functional group or absent; and

Wc is a cyclizing moiety.

According to some embodiments of the invention, X is a fatty acid residue or an ω-amino-fatty acid residue.

According to some embodiments of the invention, Y is amine or amide.

According to some embodiments of the invention, at least one of W₀, W₁, W₂, . . . Wₙ and the Z₁, Z₂, . . . Zₙ is a peptide bond.

According to some embodiments of the invention, Wc is a peptide bond.

According to some embodiments of the invention, each of the W₀, W₁, W₂, . . . Wₙ and Z₁, Z₂, . . . Z, is a peptide bond.

According to some embodiments of the invention, each of the amino acid residues is a lysine residue.

According to some embodiments of the invention, n is an integer from 3 to 10.

According to some embodiments of the invention, X is a dodecanoic acid residue and Y is an amine.

According to some embodiments of the invention, re-sensitizing effective amount of the polymer is lower than 1 MIC unit.

According to some embodiments of the invention, the re-sensitizing effective amount of the polymer ranges from ½ MIC units to ⅛ MIC unit, or from ½ MIC to ¼ MIC.

According to some embodiments of the invention, the polymer is selected from the group consisting of NC₁₂(KNC₁₂K)₂NH₂ (SEQ ID NO: 1), C₁₂₍₅₋ₑₙₑ₎KKNC₁₂KNH₂ (SEQ ID NO: 2), C₁₂K(NC₈K)₅NH₂ (SEQ ID NO: 3), C₁₂K(NC₈K)₇NH₂ (SEQ ID NO: 4), C₁₄₍₉₋ₑₙₑ₎KKNC₁₂KNH₂ (SEQ ID NO: 5), C₁₆₍₉₋ₑₙₑ₎KKNC₁₂KNH₂ (SEQ ID NO: 6), C₁₂KKNC₁₂KNH₂ (SEQ ID NO: 7), C₁₂K(KNC₁₂K)₂NH₂ (SEQ ID NO: 8), C₁₂K(KNC₁₂K)₃NH₂ (SEQ ID NO: 9) and C₁₂K(KNC₁₀K)₃NH₂ (SEQ ID NO: 10).

According to an aspect of embodiments of the invention there is provided a polymer selected from the group consisting of C₁₂₍₅₋ₑₙₑ₎KKNC₁₂KNH₂ (SEQ ID NO: 2), C₁₄₍₉₋ₑₙₑ₎KKNC₁₂KNH₂ (SEQ ID NO: 5), C₁₆₍₉₋ₑₙₑ₎KKNC₁₂KNH₂ (SEQ ID NO: 6) and C₁₂K(KNC₁₀K)₃NH₂ (SEQ ID NO: 10).

According to some embodiments of the invention, the novel polymer is being characterized as capable of re-sensitizing a pathogenic microorganism to an antimicrobial agent following a treatment of the pathogenic microorganism with the antimicrobial agent and an emergence of a resistance of the pathogenic microorganism to the antimicrobial agent.

Further according to aspects of embodiments of the invention there are provided a pharmaceutical composition comprising any of the novel polymers described herein and their use as a medicament.

According to some embodiments of the invention, the pathogenic microorganism is selected from the group consisting of Staphylococcus aureus, Pseudomonas aeruginosa, Proteus mirabilis, Stenotrophomonas maltophila, Bacillus cereus and Escherichia coli.

According to some embodiments of the invention, the antimicrobial agent is selected from the group consisting of oxacillin, piperacillin, penicillin G, ciprofloxacin, erythromycin, tetracycline, gentamicin and methicillin.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It is expected that during the life of a patent maturing from this application many relevant methods, uses, compositions and polymers will be developed and the scope of the terms methods, uses, compositions and polymers are intended to include all such new technologies a priori.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1B present comparative plots of bacterial growth of methicillin-resistant Staphylococcus aureus (MRSA 15903, a clinical isolate) versus the concentration of oxacillin (Ox, an antimicrobial agent), demonstrating that while oxacillin alone is inactive at concentrations up to 25 μM, the addition of the exemplary antimicrobial re-sensitizing polymer (OAK) NC₁₂(KNC₁₂K)₂NH₂ (SEQ ID NO: 1) (NC₁₂-2β₁₂) at sub-minimum inhibitory concentration (e.g. ⅓ and ½ MIC, when the MIC is 6.25 μM) re-sensitizes the bacteria to oxacillin (FIG. 1A), and further demonstrating that in presence of oxacillin there was merely a twofold decrease in the polymer's MIC, indicating that oxacillin does not potentiate the polymer (FIG. 1B);

FIG. 2 presents a comparative plot of the colony-forming unit (CFU) of MRSA 15903 versus incubation time, showing the sub-MIC time-kill curves obtained for oxacillin or the exemplary antimicrobial re-sensitizing polymer NC₁₂(KNC₁₂K)₂NH₂ (SEQ ID NO: 1) (NC₁₂-2β₁₂), according to some of the present embodiments, alone and in combination at low individual concentrations, further supporting the findings presented in FIGS. 1A-1B;

FIGS. 3A-3D present the results of experimental induction of oxacillin-resistance in S. aureus and re-sensitization of the bacteria by an exemplary antimicrobial re-sensitizing polymer to oxacillin (Ox), wherein FIG. 3A shows the emergence of resistance of S. aureus (ATCC 29213, an oxacillin-sensitive strain) when exposed to oxacillin alone (line 1 marked by white triangles in FIG. 3A) or to mixtures of oxacillin and sub-MIC concentrations of the antimicrobial re-sensitizing polymer (OAK) NC₁₂(KNC₁₂K)₂NH₂ (SEQ ID NO: 1) (¼ and ⅓ MIC, respectively, lines 2 and 3 marked by white and black diamonds respectively in FIG. 3A), and wherein FIGS. 3B-3D represent attempts to re-sensitize the oxacillin-resistant bacteria shown in FIG. 3A by exposing bacteria from the 15th subcultures (culture shown in line 1 in FIG. 3A corresponds to FIG. 3B, culture shown in line 2 in FIG. 3A corresponds to FIG. 3C and culture shown in line 3 in FIG. 3A corresponds to FIG. 3D) to oxacillin or polymer alone or to mixtures of oxacillin and sub-MIC concentrations of the polymer (data were obtained from at least two independent experiments performed in duplicates);

FIG. 4 presents comparative plots of bacterial growth of Staphylococcus aureus MRSA 15903 versus concentration of oxacillin (Ox) with or without potentiation by an exemplary antimicrobial re-sensitizing polymer (OAK) C₁₂₍₅₋ₑₙₑ₎KKNC₁₂KNH₂ (SEQ ID NO: 2), demonstrating that the presence of the polymer at concentrations well below its MIC value, namely ¼ MIC, endows potency to oxacillin at an optimal polymer concentration of 2.1 μM;

FIGS. 5A-5B present the results of the experimental induction of oxacillin-resistance in Staphylococcus aureus (ATCC 29213, an oxacillin-sensitive strain) and re-sensitization of the resistant bacteria, wherein FIG. 5A is a comparative plot of relative MIC of oxacillin versus the bacteria generation, showing that the relative MIC of oxacillin alone or in presence of the lowest re-sensitizing polymer concentration (¼ MIC=1.6 μM) has increased by 4 folds, reflecting emergence of resistance, unlike the effect recorded for the polymer alone or oxacillin combined with ⅓ or ½ the MIC of the exemplary re-sensitizing polymer C₁₂₍₅₋ₑₙₑ₎KKNC₁₂KNH₂ (SEQ ID NO: 2), and wherein FIG. 5B is a bar graph showing the relative MIC obtained for the oxacillin-sensitive S. aureus ATCC 29213 strain when the now-resistant strain (after 10 subcultures in presence of oxacillin) was exposed again to either oxacillin or the re-sensitizing polymer alone or to mixtures of oxacillin and sub-MIC concentrations of the polymer, demonstrating that the relative MIC of oxacillin remained 4, however using the polymer alone or mixtures of oxacillin and sub-MIC polymer concentrations decreased the relative MIC and caused re-sensitization of the bacteria;

FIGS. 6A-6D demonstrate the antimicrobial re-sensitizing effect of C₁₂K(NC₈K)₇NH₂ (SEQ ID NO: 4) and C₁₂K(NC₈K)₅NH₂ (SEQ ID NO: 3), two exemplary polymers (OAKs) according to some of the present embodiments, in combination with oxacillin (Ox), as assessed against E. coli C/14213 strain after 24 hours incubation, wherein FIGS. 6A-6B show that the polymers' activity was improved in presence of oxacillin, and wherein FIGS. 6C-6D show that while oxacillin alone was inactive against E. coli, the addition of the polymers at concentrations well below their MIC value (up to ⅛ MIC) has endowed potency to oxacillin.

### DESCRIPTION OF SPECIFIC EMBODIMENTS OF THE INVENTION

The present invention, in some embodiments thereof, relates to medicinal treatments directed at overcoming an emergence of resistance to an antimicrobial treatment, and more particularly, to use of a class of polymers which exhibit a re-sensitizing effect against antimicrobial-resistance emerged in subjects having a microbial infection, following an antimicrobial treatment.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed above, the use of the currently practiced antimicrobial agents and therapies is severely limited, mainly by the development of resistance against these antimicrobial agents.

U.S. Patent Application Nos. 20070032428, 11/234,183 and 11/500,461 and WO 2006/035431, all by one of the present inventors, which are incorporated herein by reference as if fully set forth herein, teach a novel class of antimicrobial polymeric agents which were designed to exert antimicrobial activity while being chemically and pharmaceutically stable, non-toxic and non-resistance inducing, as well as methods of preparing of these agents, pharmaceutical compositions containing same and a method of treating medical conditions associated with pathological microorganisms. These antimicrobial polymeric agents were shown to be non-hemolytic and to exhibit resistibility to plasma proteases.

The design paradigms of these antimicrobial polymeric agents were based on the knowledge which accumulated over the years on the nature of antimicrobial peptides and the limitations associated with their use, and included three key attributes, namely a flexible structure, an amphiphatic character and a net positive charge.

Thus, polymeric agents composed of a plurality of positively charged amino acid residues and one or more hydrophobic moieties in the form of ω-amino-fatty acid residues, each linking two amino acid residues and/or being attached to a terminus residue such as a fatty-acid residue or a positively charged amino acid residue, have been designed and successfully practiced as antimicrobial agents.

The present inventors have now surprisingly uncovered that such polymeric agents exhibit antimicrobial re-sensitizing activity and are further characterized advantageously as effective re-sensitizing agents at concentrations well below there own bactericidal levels (below the concentration which eradicates the microorganisms), when administered in combination with an antimicrobial agent that became ineffective during a standard antimicrobial treatment in a subject, due to the emergence of resistance thereto.

As demonstrated in the Examples section that follows, these polymeric agents were found highly effective, when administered together with an antibiotic, in eradicating resistant bacteria. These polymers were shown capable of re-sensitizing bacteria which became resistant to an antibiotic, such that when the same antibiotic is re-used, it effectively eradicates the bacteria. These polymers were also shown capable of preventing the emergence of resistance, when used in combination with an antibiotic, in microorganisms that are expected to develop resistance to the antibiotic.

These polymers are therefore highly useful in treating conditions associated with resistant bacteria, by (i) being effective when administered in combination with an antimicrobial treatment that would otherwise not be effective; (ii) being effective in preventing an emergence of resistance to an antimicrobial agent, when administered in combination with the antimicrobial agent; and (iii) being effective in re-sensitizing a microorganism to an antimicrobial agent, upon an antimicrobial treatment that resulted in emergence of resistance to the antimicrobial agent used.

Thus, according to one aspect of the present invention there is provided a method of treating a medical condition associated with a pathogenic microorganism and further associated with an emergence of antimicrobial resistance in a subject still suffering from that medical condition after being treated with an antimicrobial agent. The method is effected by administering to that subject, following the treatment with the antimicrobial agent and the emergence of antimicrobial resistance to the antimicrobial agent, a re-sensitizing effective amount of a polymer as defined, described and exemplified hereinbelow, henceforth the polymer(s) or OAK(s), thereby re-sensitizing the microorganism to the antimicrobial agent.

The method is further effected by administering to the subject a therapeutically effective amount of the antimicrobial agent.

In essence, the antimicrobial agent is re-administered (administered again after the microorganism(s) developed resistance) to the subject, with the distinction that the pathogenic microorganism is now re-sensitized towards the antimicrobial agent by the polymer.

According to some embodiments, the two components, namely the antimicrobial agent and the polymer, can be administered concomitantly or the antimicrobial agent can be administered to the subject subsequent to administration of the polymer, after the pathogenic microorganism has been re-sensitized by the antimicrobial re-sensitizing polymer.

When administered subsequently, the antimicrobial agent can be administered 10 minutes, 20 minutes, 30 minutes, 1 hour, 2 hours, 3 hours, 4 hours, 5 hours, 6 hours, 7 hours, 8 hours, 9 hours, 10 hours, 12 hours, 24 hours, and longer, after administration of the polymer.

The phrase “antimicrobial re-sensitizing activity”, as used herein in the context of the polymers according to the embodiments presented herein, defines a characteristic of the polymer which is related to three entities, namely (i) the polymer, (ii) an antimicrobial agent, and (iii) a microorganism which became or may become resistant to the antimicrobial agent in the sense that the microorganism is no longer sensitive to the antimicrobial agent. Thus, the existence on an antimicrobial re-sensitizing activity allows the polymer to endow potency to, potentiate or re-potentiate the antimicrobial agent against the microorganism by re-sensitizing the microorganism to the antimicrobial agent.

By “re-sensitizing”, it is meant that a microorganism that was sensitive (susceptible) to a treatment with antimicrobial agent and became resistant to such a treatment, is turned again to be sensitive (susceptible) to such a treatment.

As used herein, the phrase “re-sensitizing effective amount” describes an amount of the antimicrobial re-sensitizing polymer, which is sufficient to reverse the emerged resistance towards the antimicrobial agent.

In some embodiments, this phrase describes an amount of the polymer which is sufficient to reverse, or prevent, the emergence of resistance in the pathogenic microorganism causing the medical condition.

As used herein, the phrase “therapeutically effective amount” describes an amount of an active agent being administered, which will relieve to some extent one or more of the symptoms of the condition being treated.

In the context of the present embodiments, the phrase “therapeutically effective amount” describes an amount of an antimicrobial agent (including an antimicrobial polymer) being administered and/or re-administered, which will relieve to some extent one or more of the symptoms of the condition being treated by being at a level that is harmful to the target microorganism(s), namely a bactericidal level or otherwise a level that inhibits the microorganism growth or eradicates the microorganism.

It should be noted herein that a re-sensitizing effective amount with respect to the polymer, according to embodiments of the present invention, or any other agent, is substantially different than a therapeutically effective amount of the same agent in the sense that a re-sensitizing effective amount is not expected to be sufficient to cause destruction or disruption to the life-cycle of the target microorganism(s) when used exclusively, without the presence of another antimicrobial agent. The polymer may have an antimicrobial activity by its own virtue, or lack such activity altogether.

In some embodiments, the polymer as described and used herein, has an antimicrobial therapeutic activity. A re-sensitizing effective amount of such a therapeutically active polymer is typically lower than the therapeutically effective amount of that polymer when used as an antimicrobial agent against the microorganism causing the condition to be treated.

Thus, according to some embodiments of the invention, the re-sensitizing effective amount of a polymer is lower than the therapeutically effective amount of this polymer with respect to the microorganism to be eradicated if/when the polymer is administered by itself per-se.

The efficacy of an antimicrobial agent is oftentimes referred to in minimal inhibitory concentration units, or MIC units. A MIC is the lowest concentration of an antimicrobial agent, typically measured in micro-molar (μM) or micrograms per milliliter (μg/ml) units, that can inhibit the growth of a microorganism after a period of incubation, typically 24 hours. MIC values are used as diagnostic criteria to evaluate resistance of microorganisms to an antimicrobial agent, and for monitoring the activity of an antimicrobial agent in question. MICs are determined by standard laboratory methods, as these are described and demonstrated in the Examples section that follows. Standard laboratory methods typically follow a standard guideline of a reference body such as the Clinical and Laboratory Standards Institute (CLSI), British Society for Antimicrobial Chemotherapy (BSAC) or The European Committee on Antimicrobial Susceptibility Testing (EUCAST). In clinical practice, the minimum inhibitory concentrations are used to determine the amount of antibiotic agent that the subject receives as well as the type of antibiotic agent to be used.

As presented in the Examples section that follows, the polymers described herein exhibit MIC values per-se in the range of 3-7 μM. However, as antimicrobial re-sensitizing agents, the polymers described herein can be used effectively at as low as one quarter of these concentrations.

Thus, in some embodiments, a re-sensitizing effective amount of a polymer as described herein ranges from 1 MIC to ⅛ MIC. In some embodiments, the re-sensitizing effective amount ranges from ½ MIC to ¼ MIC.

Accordingly, there is provided a method of re-sensitizing a pathogenic microorganism to an antimicrobial agent, following a treatment of the pathogenic microorganism with the antimicrobial agent and a subsequent emergence of a resistance of the pathogenic microorganism to the antimicrobial agent. The method is effected by contacting the pathogenic microorganism with a re-sensitizing effective amount of the polymer(s) described herein. In the context of this aspect, the re-sensitizing effective amount is lower than the therapeutically effective amount of the polymer with respect to the pathogenic microorganism, as described herein.

According to some embodiments of the method of re-sensitizing a pathogenic microorganism presented hereinabove, contacting the microorganism with the polymer is effected by administering the re-sensitizing effective amount of the polymer to a subject having a medical condition associated with the microorganism and further associated with an emergence of antimicrobial resistance in this subject h following treatment with an antimicrobial agent. The re-sensitizing method can be further be effected by contacting the pathogenic microorganism with the antimicrobial agent, subsequent to or concomitant with the re-sensitization thereof by the polymer, as detailed herein.

According to other embodiments of the method of re-sensitizing a pathogenic microorganism presented hereinabove, administering the polymer is followed by administering the antimicrobial agent to the subject. According to embodiments of the present invention, and as stated hereinabove, the antimicrobial agent can be re-administered concomitant with or subsequent to the administration of the antimicrobial re-sensitization polymer.

In any of the methods described herein, the polymer and/or the antimicrobial agent can be administered as a part of a pharmaceutical composition, which further comprises a pharmaceutical acceptable carrier, as detailed hereinbelow.

The carrier is selected suitable to the selected route of administration.

The polymer and/or the antimicrobial agent can be administered via any administration route, including, but not limited to, orally, by inhalation, or parenterally, for example, by intravenous drip or intraperitoneal, subcutaneous, intramuscular or intravenous injection, or topically (including ophtalmically, vaginally, rectally, intranasally).

In some embodiments, the polymer is administered by intraperitoneal or subcutaneous injection.

According to another aspect of the present invention, there is provided a use of a polymer as presented herein, in the manufacture of a medicament for treating a medical condition associated with a pathogenic microorganism and further associated with an emergence of antimicrobial resistance in a subject having the medical condition and treated with an antimicrobial agent. According to this aspect, the medicament is used in combination with the antimicrobial agent and is selected such that a re-sensitizing effective amount of the polymer is used, the re-sensitizing effective amount being substantially lower than a therapeutically effective amount of the polymer with respect to the pathogenic microorganism, as described herein. As in some other aspects presented herein, and according to some embodiments, the polymer can be used in combination with the antimicrobial agent, which can then be administered concomitant with or subsequent to administering the polymer.

Accordingly, there is provided a use of a polymer as described herein in the manufacture of a medicament for re-sensitizing a pathogenic microorganism to an antimicrobial agent following a treatment of the pathogenic microorganism with the antimicrobial agent and a subsequent emergence of a resistance of the pathogenic microorganism to the antimicrobial, wherein a re-sensitizing effective amount of the polymer is used, the re-sensitizing effective amount being lower than a therapeutically effective amount of the polymer with respect to the pathogenic microorganism. Also in this aspect and according to some embodiments, the polymer can be used in combination with the antimicrobial agent, which can then be administered concomitant with or subsequent to administering the polymer.

Hence, according to another aspect of embodiments of the invention, there is provided a pharmaceutical composition which comprises, as active ingredients, one or more of the antimicrobial re-sensitizing polymers presented herein, one or more antimicrobial agents and a pharmaceutically acceptable carrier. According to some embodiments, the composition is packaged in a packaging material and identified in print, in or on the packaging material, for use in the treatment of a medical condition associated with a pathogenic microorganism and further associated with an emergence of antimicrobial resistance in a subject having the medical condition and treated with an antimicrobial agent.

As used herein the phrase “pharmaceutical composition” or the term “medicament” refer to a preparation of the antimicrobial re-sensitizing polymer described herein, with other chemical components such as pharmaceutically acceptable and suitable carriers and excipients, and optionally with additional active agents, such as an antimicrobial agent. The purpose of a pharmaceutical composition is to facilitate administration of a compound to a subject.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

The pharmaceutical composition may be formulated for administration in either one or more of routes depending on whether local or systemic treatment or administration is of choice, and on the area to be treated. Administration may be done orally, by inhalation, or parenterally, for example by intravenous drip or intraperitoneal, subcutaneous, intramuscular or intravenous injection, or topically (including ophtalmically, vaginally, rectally, intranasally).

Formulations for topical administration may include but are not limited to lotions, ointments, gels, creams, suppositories, drops, liquids, sprays and powders. Conventional pharmaceutical carriers, aqueous, powder or oily bases, thickeners and the like may be necessary or desirable.

Compositions for oral administration include powders or granules, suspensions or solutions in water or non-aqueous media, sachets, pills, caplets, capsules or tablets. Thickeners, diluents, flavorings, dispersing aids, emulsifiers or binders may be desirable.

Formulations for parenteral administration may include, but are not limited to, sterile solutions which may also contain buffers, diluents and other suitable additives. Slow release compositions are envisaged for treatment.

The amount of a composition to be administered will, of course, be dependent on the subject being treated, the severity of the affliction, the manner of administration, the judgment of the prescribing physician, etc.

Pharmaceutical compositions for use in accordance with embodiments of the invention thus may be formulated in conventional manner using one or more pharmaceutically acceptable carriers comprising excipients and auxiliaries, which facilitate processing of the polymers and antimicrobial agents into preparations which can be used pharmaceutically. Proper formulation is dependent upon the route of administration chosen. Toxicity and therapeutic efficacy of the antimicrobial agents and re-sensitizing efficacy of the polymers described herein can be determined by standard pharmaceutical procedures in experimental animals, e.g., by determining the EC₅₀, the IC₅₀ and the LD₅₀ (lethal dose causing death in 50% of the tested animals) for a subject combination of antimicrobial agent(s) and polymer(s). The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1). In general, the dosage is related to the efficacy of the active ingredient which, in the context of embodiments of the invention, is related to its minimal inhibitory concentration (MIC) and the particular pharmacokinetics and pharmacology thereof for absorption, distribution, metabolism, excretion and toxicity (ADME-Tox) parameters. For antimicrobial agents, a therapeutically effective amount is oftentimes about ten-fold the MIC of the antimicrobial agent. The re-sensitization effective amount for a polymer may be a low as equal or less than one MIC unit.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a polymer, either alone or in combination with an antimicrobial agent, formulated in a compatible pharmaceutical carrier may also be prepared, placed in an appropriate container, and labeled for treatment of an indicated condition, as is detailed herein.

As presented hereinabove, antimicrobial re-sensitizing polymers are directed at uses in combination with antimicrobial agents, and as further presented, the two active components may be administered concomitantly or sequentially as separate compositions. Hence, there is an advantage in providing the health-care provider or the self-administering subject a kit which will include all the required compositions in one package.

Thus, according to yet another aspect of the present invention, there is provided a pharmaceutical kit which includes inside a packaging material a polymer as described herein and an anti-microbial agent being individually packaged. The kit can then be labeled according to its intended use, such as for treating a medical condition associated with a pathogenic microorganism and further associated with an emergence of antimicrobial resistance in a subject having the medical condition and treated with an antimicrobial agent, and/or for re-sensitizing a pathogenic microorganism to an antimicrobial agent, following a treatment of the pathogenic microorganism with the antimicrobial agent and a subsequent emergence of a resistance of the pathogenic microorganism to the antimicrobial.

As described hereinabove, the polymers described herein have unique features that enable to use these polymers as antimicrobial re-sensitization agents as dosages that are lower than the dosages commonly practiced with common antimicrobial agents.

Hence, according to another aspect of embodiments of the invention, there is provided a pharmaceutical composition unit dosage form which includes a re-sensitizing effective amount of a polymer as described herein. According to this aspect, the re-sensitizing effective amount is selected such that it effects a re-sensitization of a pathogenic microorganism to an antimicrobial agent, following a treatment of the pathogenic microorganism with the antimicrobial agent and a subsequent emergence of a resistance of the pathogenic microorganism to the antimicrobial agent, wherein the re-sensitizing effective amount is lower than a therapeutically effective amount of the polymer with respect to the pathogenic microorganism.

The term “unit dosage form”, as used herein, describes physically discrete units, each unit containing a predetermined quantity of one or more active ingredient(s) calculated to produce the desired re-sensitizing effect, in association with at least one pharmaceutically acceptable carrier, diluent, excipient, or combination thereof.

The single unit dosage forms described herein can be formulated for oral, mucosal (e.g., nasal, sublingual, vaginal, buccal, or rectal), parenteral (e.g., intraperitoneal, subcutaneous, intravenous, bolus injection, intramuscular, or intraarterial), or transdermal administration to a patient. Examples of unit dosage forms include, but are not limited to: tablets including orally dissolving tablets; thin films; gelcaps; caplets; granules, capsules, such as soft elastic gelatin capsules; cachets; troches; lozenges; dispersions; suppositories; enemas; pessary; vaginal tablets; ointments; cataplasms (poultices); pastes; powders; dressings; creams; plasters; solutions; patches; liquid sprays; metered and unmetered aerosols (e.g., nasal sprays or inhalers); drops; lyophilized compositions; transdermal patches; gels; liquid dosage forms suitable for oral or mucosal administration to a patient, including suspensions (e.g., aqueous or non-aqueous liquid suspensions, oil-in-water emulsions, or a water-in-oil liquid emulsions), solutions, tinctures and elixirs; syrups, liquid dosage forms suitable for parenteral administration to a patient (e.g., ampoules, sterile bags); sterile solids (e.g., crystalline or amorphous solids) that can be reconstituted to provide liquid dosage forms suitable for parenteral administration to a patient; and as components of autoinjector devices.

In some embodiments, the amount of the polymer in the unit dosage form ranges from about 1 MIC units to about ⅛ MIC units, as described herein, of the polymer. In some embodiments, the pharmaceutical composition unit dosage form described herein comprises an amount of the polymer which is equal or lower than its MIC. In other embodiments, the unit dosage form comprises an amount of the polymer that is 1 MIC unit, ¾ MIC unit, ⅔ MIC unit, ½ MIC unit, ⅓ MIC unit, ¼ MIC unit and even as low as ⅛ MIC unit.

Herein throughout, the phrase “pathogenic microorganism” is used to describe any microorganism which can cause a disease or disorder in a higher organism, such as mammals in general and a human in particular. The pathogenic microorganism may belong to any family of organisms such as, but not limited to prokaryotic organisms, eubacterium, archaebacterium, eukaryotic organisms, yeast, fungi, algae, protozoan, and other parasites. Non-limiting examples of pathogenic microorganism are Plasmodium falciparum and related malaria-causing protozoan parasites, Acanthamoeba and other free-living amoebae, Aeromonas hydrophila, Anisakis and related worms, and further include, but not limited to Acinetobacter baumanii, Ascaris lumbricoides, Bacillus cereus, Brevundimonas diminuta, Campylobacter jejuni, Clostridium botulinum, Clostridium perfringens, Cryptosporidium parvum, Cyclospora cayetanensis, Diphyllobothrium, Entamoeba histolytica, certain strains of Escherichia coli, Eustrongylides, Giardia lamblia, Klebsiella pneumoniae, Listeria monocytogenes, Nanophyetus, Plesiomonas shigelloides, Proteus mirabilis, Pseudomonas aeruginosa, Salmonella, Serratia odorifera, Shigella, Staphylococcus aureus, Stenotrophomonas maltophilia, Streptococcus, Trichuris trichiura, Vibrio cholerae, Vibrio parahaemolyticus, Vibrio vulnificus and other vibrios, Yersinia enterocolitica, Yersinia pseudotuberculosis and Yersinia kristensenii.

Accordingly, a condition associated with a pathogenic microorganism describes an infectious condition that results from the presence of the microorganism in a subject. The infectious condition can be, for example, a bacterial infection, a fungal infection, a protozoal infection, and the like.

Treating a condition associated with a pathogenic microorganism describes means for preventing, reducing, ameliorating or abolishing symptoms of the infectious condition. The treatment is effected typically by inhibiting the growth and/or eradicating the pathogenic microorganism.

The phrase “antimicrobial agent”, as used herein, excludes polymers according to the embodiments of the present invention, and encompasses all other antimicrobial agents. According to the definition of microorganism presented hereinabove, the phrase “antimicrobial agent” encompasses antibiotic agents (also referred to herein as antibiotic) as well as anti-fungal, anti-protozoan, anti-parasitic agents and like.

According to some embodiments, the antimicrobial agent is an antibiotic agent. In general, but without being bound to any particular theory, the mechanism of the antimicrobial activity of an antimicrobial agent, according to the embodiments of the present invention, is different that the mechanism of the activity of the polymers, according to the embodiments of the present invention.

Non-limiting examples of antimicrobial agents that are suitable for use in this context of the present invention include, without limitation, mandelic acid, 2,4-dichlorobenzenemethanol, 4-[bis(ethylthio)methyl]-2-methoxyphenol, 4-epi-tetracycline, 4-hexylresorcinol, 5,12-dihydro-5,7,12,14-tetrazapentacen, 5-chlorocarvacrol, 8-hydroxyquinoline, acetarsol, acetylkitasamycin, acriflavin, alatrofloxacin, ambazon, amfomycin, amikacin, amikacin sulfate, aminoacridine, aminosalicylate calcium, aminosalicylate sodium, amino salicylic acid, ammoniumsulfobituminat, amorolfin, amoxicillin, amoxicillin sodium, amoxicillin trihydrate, amoxicillin-potassium clavulanate combination, amphotericin B, ampicillin, ampicillin sodium, ampicillin trihydrate, ampicillin-sulbactam, apalcillin, arbekacin, aspoxicillin, astromicin, astromicin sulfate, azanidazole, azidamfenicol, azidocillin, azithromycin, azlocillin, aztreonam, bacampicillin, bacitracin, bacitracin zinc, bekanamycin, benzalkonium, benzethonium chloride, benzoxonium chloride, berberine hydrochloride, biapenem, bibrocathol, biclotymol, bifonazole, bismuth subsalicylate, bleomycin antibiotic complex, bleomycin hydrochloride, bleomycin sulfate, brodimoprim, bromochlorosalicylanilide, bronopol, broxyquinolin, butenafine, butenafine hydrochloride, butoconazol, calcium undecylenate, candicidin antibiotic complex, capreomycin, carbenicillin, carbenicillin disodium, carfecillin, carindacillin, carumonam, carzinophilin, caspofungin acetate, cefacetril, cefaclor, cefadroxil, cefalexin, cefalexin hydrochloride, cefalexin sodium, cefaloglycin, cefaloridine, cefalotin, cefalotin sodium, cefamandole, cefamandole nafate, cefamandole sodium, cefapirin, cefapirin sodium, cefatrizine, cefatrizine propylene glycol, cefazedone, cefazedone sodium salt, cefazolin, cefazolin sodium, cefbuperazone, cefbuperazone sodium, cefcapene, cefcapene pivoxil hydrochloride, cefdinir, cefditoren, cefditoren pivoxil, cefepime, cefepime hydrochloride, cefetamet, cefetamet pivoxil, cefixime, cefmenoxime, cefmetazole, cefmetazole sodium, cefminox, cefminox sodium, cefmolexin, cefodizime, cefodizime sodium, cefonicid, cefonicid sodium, cefoperazone, cefoperazone sodium, ceforanide, cefoselis sulfate, cefotaxime, cefotaxime sodium, cefotetan, cefotetan disodium, cefotiam, cefotiam hexetil hydrochloride, cefotiam hydrochloride, cefoxitin, cefoxitin sodium, cefozopran hydrochloride, cefpiramide, cefpiramide sodium, cefpirome, cefpirome sulfate, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftizoxime sodium, ceftriaxone, ceftriaxone sodium, cefuroxime, cefuroxime axetil, cefuroxime sodium, cetalkonium chloride, cetrimide, cetrimonium, cetylpyridinium, chloramine T, chloramphenicol, chloramphenicol palmitate, chloramphenicol succinate sodium, chlorhexidine, chlormidazole, chlormidazole hydrochloride, chloroxylenol, chlorphenesin, chlorquinaldol, chlortetracycline, chlortetracycline hydrochloride, ciclacillin, ciclopirox, cinoxacin, ciprofloxacin, ciprofloxacin hydrochloride, citric acid, clarithromycin, clavulanate potassium, clavulanate sodium, clavulanic acid, clindamycin, clindamycin hydrochloride, clindamycin palmitate hydrochloride, clindamycin phosphate, clioquinol, cloconazole, cloconazole monohydrochloride, clofazimine, clofoctol, clometocillin, clomocycline, clotrimazol, cloxacillin, cloxacillin sodium, colistin, colistin sodium methanesulfonate, colistin sulfate, cycloserine, dactinomycin, danofloxacin, dapsone, daptomycin, daunorubicin, DDT, demeclocycline, demeclocycline hydrochloride, dequalinium, dibekacin, dibekacin sulfate, dibrompropamidine, dichlorophene, dicloxacillin, dicloxacillin sodium, didecyldimethylammonium chloride, dihydrostreptomycin, dihydrostreptomycin sulfate, diiodohydroxyquinolin, dimetridazole, dipyrithione, dirithromycin, DL-menthol, D-menthol, dodecyltriphenylphosphonium bromide, doxorubicin, doxorubicin hydrochloride, doxycycline, doxycycline hydrochloride, econazole, econazole nitrate, enilconazole, enoxacin, enrofloxacin, eosine, epicillin, ertapenem sodium, erythromycin, erythromycin estolate, erythromycin ethyl succinate, erythromycin lactobionate, erythromycin stearate, ethacridine, ethacridine lactate, ethambutol, ethanoic acid, ethionamide, ethyl alcohol, eugenol, exalamide, faropenem, fenticonazole, fenticonazole nitrate, fezatione, fleroxacin, flomoxef, flomoxef sodium, florfenicol, flucloxacillin, flucloxacillin magnesium, flucloxacillin sodium, fluconazole, flucytosine, flumequine, flurithromycin, flutrimazole, fosfomycin, fosfomycin calcium, fosfomycin sodium, framycetin, framycetin sulphate, furagin, furazolidone, fusafungin, fusidic acid, fusidic acid sodium salt, gatifloxacin, gemifloxacin, gentamicin antibiotic complex, gentamicin c1a, gentamycin sulfate, glutaraldehyde, gramicidin, grepafloxacin, griseofulvin, halazon, haloprogine, hetacillin, hetacillin potassium, hexachlorophene, hexamidine, hexetidine, hydrargaphene, hydroquinone, hygromycin, imipenem, isepamicin, isepamicin sulfate, isoconazole, isoconazole nitrate, isoniazid, isopropanol, itraconazole, josamycin, josamycin propionate, kanamycin, kanamycin sulphate, ketoconazole, kitasamycin, lactic acid, lanoconazole, lenampicillin, leucomycin A1, leucomycin A13, leucomycin A4, leucomycin A5, leucomycin A6, leucomycin A7, leucomycin A8, leucomycin A9, levofloxacin, lincomycin, lincomycin hydrochloride, linezolid, liranaftate, 1-menthol, lomefloxacin, lomefloxacin hydrochloride, loracarbef, lymecyclin, lysozyme, mafenide acetate, magnesium monoperoxophthalate hexahydrate, mecetronium ethylsulfate, mecillinam, meclocycline, meclocycline sulfosalicylate, mepartricin, merbromin, meropenem, metalkonium chloride, metampicillin, methacycline, methenamin, methyl salicylate, methylbenzethonium chloride, methylrosanilinium chloride, meticillin, meticillin sodium, metronidazole, metronidazole benzoate, mezlocillin, mezlocillin sodium, miconazole, miconazole nitrate, micronomicin, micronomicin sulfate, midecamycin, minocycline, minocycline hydrochloride, miocamycin, miristalkonium chloride, mitomycin c, monensin, monensin sodium, morinamide, moxalactam, moxalactam disodium, moxifloxacin, mupirocin, mupirocin calcium, nadifloxacin, nafcillin, nafcillin sodium, naftifine, nalidixic acid, natamycin, neomycin a, neomycin antibiotic complex, neomycin C, neomycin sulfate, neticonazole, netilmicin, netilmicin sulfate, nifuratel, nifuroxazide, nifurtoinol, nifurzide, nimorazole, niridazole, nitrofurantoin, nitrofurazone, nitroxolin, norfloxacin, novobiocin, nystatin antibiotic complex, octenidine, ofloxacin, oleandomycin, omoconazol, orbifloxacin, ornidazole, ortho-phenylphenol, oxacillin, oxacillin sodium, oxiconazole, oxiconazole nitrate, oxoferin, oxolinic acid, oxychlorosene, oxytetracycline, oxytetracycline calcium, oxytetracycline hydrochloride, panipenem, paromomycin, paromomycin sulfate, pazufloxacine, pefloxacin, pefloxacin mesylate, penamecillin, penicillin G, penicillin G potassium, penicillin G sodium, penicillin V, penicillin V calcium, penicillin V potassium, pentamidine, pentamidine diisetionate, pentamidine mesilas, pentamycin, phenethicillin, phenol, phenoxyethanol, phenylmercuriborat, PHMB, phthalylsulfathiazole, picloxydin, pipemidic acid, piperacillin, piperacillin sodium, pipercillin sodium-tazobactam sodium, piromidic acid, pivampicillin, pivcefalexin, pivmecillinam, pivmecillinam hydrochloride, policresulen, polymyxin antibiotic complex, polymyxin B, polymyxin B sulfate, polymyxin B1, polynoxylin, povidone-iodine, propamidin, propenidazole, propicillin, propicillin potassium, propionic acid, prothionamide, protiofate, pyrazinamide, pyrimethamine, pyrithion, pyrrolnitrin, quinoline, quinupristin-dalfopristin, resorcinol, ribostamycin, ribostamycin sulfate, rifabutin, rifampicin, rifamycin, rifapentine, rifaximin, ritiometan, rokitamycin, rolitetracycline, rosoxacin, roxithromycin, rufloxacin, salicylic acid, secnidazol, selenium disulphide, sertaconazole, sertaconazole nitrate, siccanin, sisomicin, sisomicin sulfate, sodium thiosulfate, sparfloxacin, spectinomycin, spectinomycin hydrochloride, spiramycin antibiotic complex, spiramycin b, streptomycin, streptomycin sulphate, succinylsulfathiazole, sulbactam, sulbactam sodium, sulbenicillin disodium, sulbentin, sulconazole, sulconazole nitrate, sulfabenzamide, sulfacarbamide, sulfacetamide, sulfacetamide sodium, sulfachlorpyridazine, sulfadiazine, sulfadiazine silver, sulfadiazine sodium, sulfadicramide, sulfadimethoxine, sulfadoxine, sulfaguanidine, sulfalene, sulfamazone, sulfamerazine, sulfamethazine, sulfamethazine sodium, sulfamethizole, sulfamethoxazole, sulfamethoxazol-trimethoprim, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, sulfonamides, sultamicillin, sultamicillin tosilate, tacrolimus, talampicillin hydrochloride, teicoplanin A2 complex, teicoplanin A2-1, teicoplanin A2-2, teicoplanin A2-3, teicoplanin A2-4, teicoplanin A2-5, teicoplanin A3, teicoplanin antibiotic complex, telithromycin, temafloxacin, temocillin, tenoic acid, terbinafine, terconazole, terizidone, tetracycline, tetracycline hydrochloride, tetracycline metaphosphate, tetramethylthiuram monosulfide, tetroxoprim, thiabendazole, thiamphenicol, thiaphenicol glycinate hydrochloride, thiomersal, thiram, thymol, tibezonium iodide, ticarcillin, ticarcillin-clavulanic acid mixture, ticarcillin disodium, ticarcillin monosodium, tilbroquinol, tilmicosin, tinidazole, tioconazole, tobramycin, tobramycin sulfate, tolciclate, tolindate, tolnaftate, toloconium metilsulfat, toltrazuril, tosufloxacin, triclocarban, triclosan, trimethoprim, trimethoprim sulfate, triphenylstibinsulfide, troleandomycin, trovafloxacin, tylosin, tyrothricin, undecoylium chloride, undecylenic acid, vancomycin, vancomycin hydrochloride, viomycin, virginiamycin antibiotic complex, voriconazol, xantocillin, xibornol and zinc undecylenate.

In some embodiments, the antimicrobial agent is an antibiotic. Exemplary antibiotics include, but are not limited to oxacillin, piperacillin, penicillin G, ciprofloxacin, erythromycin, tetracycline, gentamicin and methicillin. These antibiotics are known to be associated with emergence of resistance thereto.

According to some embodiments, the polymer of any aspect described herein is composed of a plurality of positively charged amino acid residues and at least one ω-amino-fatty acid residue, as these terms are defined hereinbelow, wherein the ω-amino-fatty acid residue is being covalently linked to at least two amino acid residues in the sequence of the polymer via the N-alpha of one amino acid residue and via the C-alpha of the other amino acid residue in the sequence via a peptide bond.

According to some embodiments, the polymer can be a linear polymer or a cyclic polymer, as these terms are defined hereinbelow. As specified hereinabove, each of the polymers, according to embodiments of the invention, comprises two or more monomers, also referred to herein interchangeably as residues, therefore, the polymers described herein each is comprised of a linear or cyclic chain made of a sequence of positively charged amino acid residues, interrupted by one or more ω-amino-fatty acid residues.

The present embodiments further encompass methods and compositions using any enantiomers, prodrugs, solvates, hydrates and/or pharmaceutically acceptable salts of the polymers described herein.

As used herein, the term “enantiomer” refers to a stereoisomer of a polymer that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems.

The term “prodrug” refers to an agent, which is converted into the active polymer (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the polymer as described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The phrase “pharmaceutically acceptable salt” refers to a charged species of the parent polymer and its counter ion, which is typically used to modify the solubility characteristics of the parent compound and/or to reduce any significant irritation to an organism by the parent polymer, while not abrogating the biological activity and properties of the administered polymer. An example, without limitation, of a pharmaceutically acceptable salt would be a carboxylate anion and a cation such as, but not limited to, ammonium, sodium, potassium and the like.

As used herein throughout the term “amino acid” or “amino acids” is understood to include the 20 genetically coded amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids and other non-naturally occurring amino acids.

Tables 1 and 2 below list the genetically encoded amino acids (Table 1) and non-limiting examples of non-conventional/modified amino acids (Table 2) which can be used with the present invention.

TABLE 1

| Amino acid | Three-Letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane-carboxylate | Cpro | L-N-methylasparagine | Nmasn |
| aminoisobutyric acid | Aib | L-N-methylaspartic acid | Nmasp |
| aminonorbornyl-carboxylate | Norb | L-N-methylcysteine | Nmcys |
| Cyclohexylalanine | Chexa | L-N-methylglutamine | Nmgin |
| Cyclopentylalanine | Cpen | L-N-methylglutamic acid | Nmglu |
| D-alanine | Dal | L-N-methylhistidine | Nmhis |
| D-arginine | Darg | L-N-methylisolleucine | Nmile |
| D-aspartic acid | Dasp | L-N-methylleucine | Nmleu |
| D-cysteine | Dcys | L-N-methyllysine | Nmlys |
| D-glutamine | Dgln | L-N-methylmethionine | Nmmet |
| D-glutamic acid | Dglu | L-N-methylnorleucine | Nmnle |
| D-histidine | Dhis | L-N-methylnorvaline | Nmnva |
| D-isoleucine | Dile | L-N-methylornithine | Nmorn |
| D-leucine | Dleu | L-N-methylphenylalanine | Nmphe |
| D-lysine | Dlys | L-N-methylproline | Nmpro |
| D-methionine | Dmet | L-N-methylserine | Nmser |
| D/L-ornithine | D/Lorn | L-N-methylthreonine | Nmthr |
| D-phenylalanine | Dphe | L-N-methyltryptophan | Nmtrp |
| D-proline | Dpro | L-N-methyltyrosine | Nmtyr |
| D-serine | Dser | L-N-methylvaline | Nmval |
| D-threonine | Dthr | L-N-methylethylglycine | Nmetg |
| D-tryptophan | Dtrp | L-N-methyl-t-butylglycine | Nmtbug |
| D-tyrosine | Dtyr | L-norleucine | Nle |
| D-valine | Dval | L-norvaline | Nva |
| D-α-methylalanine | Dmala | α-methyl-aminoisobutyrate | Maib |
| D-α-methylarginine | Dmarg | α-methyl-γ-aminobutyrate | Mgabu |
| D-α-methylasparagine | Dmasn | α-methylcyclohexylalanine | Mchexa |
| D-α-methylaspartate | Dmasp | α-methylcyclopentylalanine | Mcpen |
| D-α-methylcysteine | Dmcys | α-methyl-α-napthylalanine | Manap |
| D-α-methylglutamine | Dmgln | α-methylpenicillamine | Mpen |
| D-α-methylhistidine | Dmhis | N-(4-aminobutyl)glycine | Nglu |
| D-α-methylisoleucine | Dmile | N-(2-aminoethyl)glycine | Naeg |
| D-α-methylleucine | Dmleu | N-(3-aminopropyl)glycine | Norn |
| D-α-methyllysine | Dmlys | N-amino-a-methylbutyrate | Nmaabu |
| D-α-methylmethionine | Dmmet | α-napthylalanine | Anap |
| D-α-methylornithine | Dmorn | N-benzylglycine | Nphe |
| D-α-methylphenylalanine | Dmphe | N-(2-carbamylethyl)glycine | Ngln |
| D-α-methylproline | Dmpro | N-(carbamylmethyl)glycine | Nasn |
| D-α-methylserine | Dmser | N-(2-carboxyethyl)glycine | Nglu |
| D-α-methylthreonine | Dmthr | N-(carboxymethyl)glycine | Nasp |
| D-α-methyltryptophan | Dmtrp | N-cyclobutylglycine | Ncbut |
| D-α-methyltyrosine | Dmty | N-cycloheptylglycine | Nchep |
| D-α-methylvaline | Dmval | N-cyclohexylglycine | Nchex |
| D-α-methylalnine | Dnmala | N-cyclodecylglycine | Ncdec |
| D-α-methylarginine | Dnmarg | N-cyclododeclglycine | Ncdod |
| D-α-methylasparagine | Dnmasn | N-cyclooctylglycine | Ncoct |
| D-α-methylasparatate | Dnmasp | N-cyclopropylglycine | Ncpro |
| D-α-methylcysteine | Dnmcys | N-cycloundecylglycine | Ncund |
| D-N-methylleucine | Dnmleu | N-(2,2-diphenylethyl)glycine | Nbhm |
| D-N-methyllysine | Dnmlys | N-(3,3-diphenylpropyl)glycine | Nbhe |
| N-methylcyclohexylalanine | Nmchexa | N-(3-indolylyethyl)glycine | Nhtrp |
| D-N-methylornithine | Dnmorn | N-methyl-γ-aminobutyrate | Nmgabu |
| N-methylglycine | Nala | D-N-methylmethionine | Dnmmet |
| N-methylaminoisobutyrate | Nmaib | N-methylcyclopentylalanine | Nmcpen |
| N-(1-methylpropyl)glycine | Nile | D-N-methylphenylalanine | Dnmphe |
| N-(2-methylpropyl)glycine | Nile | D-N-methylproline | Dnmpro |
| N-(2-methylpropyl)glycine | Nleu | D-N-methylserine | Dnmser |
| D-N-methyltryptophan | Dnmtrp | D-N-methylserine | Dnmser |
| D-N-methyltyrosine | Dnmtyr | D-N-methylthreonine | Dnmthr |
| D-N-methylvaline | Dnmval | N-(1-methylethyl)glycine | Nva |
| γ-aminobutyric acid | Gabu | N-methyla-napthylalanine | Nmanap |
| L-t-butylglycine | Tbug | N-methylpenicillamine | Nmpen |
| L-ethylglycine | Etg | N-(p-hydroxyphenyl)glycine | Nhtyr |
| L-homophenylalanine | Hphe | N-(thiomethyl)glycine | Ncys |
| L-α-methylarginine | Marg | penicillamine | Pen |
| L-α-methylaspartate | Masp | L-α-methylalanine | Mala |
| L-α-methylcysteine | Mcys | L-α-methylasparagine | Masn |
| L-α-methylglutamine | Mgln | L-α-methyl-t-butylglycine | Mtbug |
| L-α-methylhistidine | Mhis | L-methylethylglycine | Metg |
| L-α-methylisoleucine | Mile | L-α-methylglutamate | Mglu |
| D-N-methylglutamine | Dnmgln | L-α-methylhomo phenylalanine | Mhphe |
| D-N-methylglutamate | Dnmglu | N-(2-methylthioethyl)glycine | Nmet |
| D-N-methylhistidine | Dnmhis | N-(3-guanidinopropyl)glycine | Narg |
| D-N-methylisoleucine | Dnmile | N-(1-hydroxyethyl)glycine | Nthr |
| D-N-methylleucine | Dnmleu | N-(hydroxyethyl)glycine | Nser |
| D-N-methyllysine | Dnmlys | N-(imidazolylethyl)glycine | Nhis |
| N-methylcyclohexylalanine | Nmchexa | N-(3-indolylyethyl)glycine | Nhtrp |
| D-N-methylornithine | Dnmorn | N-methyl-γ-aminobutyrate | Nmgabu |
| N-methylglycine | Nala | D-N-methylmethionine | Dnmmet |
| N-methylaminoisobutyrate | Nmaib | N-methylcyclopentylalanine | Nmcpen |
| N-(1-methylpropyl)glycine | Nile | D-N-methylphenylalanine | Dnmphe |
| N-(2-methylpropyl)glycine | Nleu | D-N-methylproline | Dnmpro |
| D-N-methyltryptophan | Dnmtrp | D-N-methylserine | Dnmser |
| D-N-methyltyrosine | Dnmtyr | D-N-methylthreonine | Dnmthr |
| D-N-methylvaline | Dnmval | N-(1-methylethyl)glycine | Nval |
| γ-aminobutyric acid | Gabu | N-methyla-napthylalanine | Nmanap |
| L-t-butylglycine | Tbug | N-methylpenicillamine | Nmpen |
| L-ethylglycine | Etg | N-(p-hydroxyphenyl)glycine | Nhtyr |
| L-homophenylalanine | Hphe | N-(thiomethyl)glycine | Ncys |
| L-α-methylarginine | Marg | penicillamine | Pen |
| L-α-methylaspartate | Masp | L-α-methylalanine | Mala |
| L-α-methylcysteine | Mcys | L-α-methylasparagine | Masn |
| L-α-methylglutamine | Mgln | L-α-methyl-t-butylglycine | Mtbug |
| L-α-methylhistidine | Mhis | L-methylethylglycine | Metg |
| L-α-methylisoleucine | Mile | L-α-methylglutamate | Mglu |
| L-α-methylleucine | Mleu | L-α-methylhomophenylalanine | Mhphe |
| L-α-methylmethionine | Mmet | N-(2-methylthioethyl)glycine | Nmet |
| L-α-methylnorvaline | Mnva | L-α-methyllysine | Mlys |
| L-α-methylphenylalanine | Mphe | L-α-methylnorleucine | Mnle |
| L-α-methylserine | mser | L-α-methylornithine | Morn |
| L-α-methylvaline | Mtrp | L-α-methylproline | Mpro |
| L-α-methylleucine | Mval Nnbhm | L-α-methylthreonine | Mthr |
| N-(N-(2,2-diphenylethyl)carbamylmethyl- | Nnbhm | L-α-methyltyrosine | Mtyr |
| glycine | | | |
| 1-carboxy-1-(2,2-diphenyl | Nmbc | L-N-methylhomophenylalanine | Nmhphe |
| ethylamino)cyclopropane | | | |
| N-(N-(3,3- | Nnbhe | D/L-citrulline | D/Lctr |
| diphenylpropyl)carbamylmethyl(1)glycine | | | |

As is well accepted in the art in the molecular context, the term “residue”, as used herein, refers to a portion, and typically a major portion of a molecular entity, such as molecule or a part of a molecule such as a group, which has underwent a chemical reaction and is now covalently linked to another molecular entity. In the context of the present embodiments, a residue is an equivalent term to a monomeric unit within the polymer. For example, the molecular entity can be an amino acid molecule, and the portion of the amino acid which forms a part of a polypeptide chain (a polymer) after the formation of the polypeptide chain, is an amino acid residue (a monomer). An amino acid residue is therefore that part of an amino acid which is present in a peptide sequence upon reaction of, for example, an alpha-amine group thereof with a carboxylic group of an adjacent amino acid in the peptide sequence, to form a peptide amide bond and/or of an alpha-carboxylic acid group thereof with an alpha-amine group of an adjacent amino acid in the peptide sequence, to form a peptide amide bond. Similarly, the term “residue” refers to the major part of a hydrophobic moiety, such as, for example the acyl part of a fatty acid or the hydrocarbon chain in an ω-amino-fatty acid.

As used herein, the phrase “moiety” describes a part, and preferably a major part of a chemical entity or compound, which typically has certain functionality or distinguishing features.

As used herein, the phrase “hydrophobic moiety” describes a chemical moiety that has a minor or no affinity to water, that is, which has a low or no dissolvability in water and often in other polar solvents. Exemplary suitable hydrophobic moieties for use in the context of the present embodiments, include, without limitation, hydrophobic moieties that consist predominantly of one or more saturated or unsaturated, branched or unbranched hydrocarbon chains and/or aromatic rings, and one or more functional groups which may be non-hydrophobic, but do not nullify the overall hydrophobicity of the hydrophobic moiety. Representative examples include, without limitation, fatty acids, ω-amino-fatty acids, hydrophobic amino acids (amino acids with hydrophobic side-chains), alkanes, alkenes, aryls and the likes, as these terms are defined herein, and any combination thereof.

The term “side-chain”, as used herein with reference to amino acids, refers to a chemical group which is attached to the α-carbon atom of an amino acid. The side-chain is unique for each type of amino acid and typically does not take part in forming the peptide bond in a naturally occurring protein or polypeptide, but can be used to form a link between monomers in the polymer presented herein in cases the side-chain comprises a suitable functional group. For example, the side chain for glycine is hydrogen, for alanine it is methyl, for valine it is isopropyl, for phenylalanine it is benzyl, and the side chain for lysine can be regarded as an amino-butyl group, e.g., having an available amine group. For the specific side chains of all amino acids reference is made to A. L. Lehninger's text on Biochemistry (see, chapter 4).

The net positive charge of the polymer, which is one of the key characteristics of AMPs which were found to be linked to their activity, is maintained by having one or more positively charged amino acid residues in the polymer, optionally in addition to the positively charged N-terminus amine.

As used herein the phrase “positively charged amino acid” describes a hydrophilic amino acid with a side chain pKa value of greater than 7, namely a basic amino acid. Basic amino acids typically have positively charged side chains at physiological pH due to association with a hydronium ion. Naturally occurring (genetically encoded) basic amino acids include lysine (Lys, K), arginine (Arg, R) and histidine (His, H), while non-natural (non-genetically encoded, or non-standard) basic amino acids include, for example, ornithine, 2,3,-diaminopropionic acid, 2,4-diaminobutyric acid, 2,5,6-triaminohexanoic acid, 2-amino-4-guanidinobutanoic acid, and homoarginine.

In some embodiments, all the amino acid residues in the polymer are positively charged amino acid residues. Exemplary polymers according to some embodiment include a plurality of lysine residues.

The hydrophobic moieties that are used in the context of this and other preferred embodiments have one or more hydrocarbon chains, and are capable of linking to one or two other components in the polymer (e.g., one or two of an amino acid residue and another hydrophobic moiety) via two peptide bonds. These moieties therefore preferably have a carboxylic group at one end of the hydrocarbon chain (for linking a free amine group) and an amine group at the other (for linking a carboxylic acid group).

The hydrocarbon chain connecting the carboxylic and amine groups in such a hydrophobic moiety preferably has from 4 to 30 carbon atoms.

In some embodiments of the present invention, the hydrophobic moiety residue is a fatty acid residue wherein the hydrocarbon chain can be unbranched and saturated, branched and saturated, unbranched and unsaturated or branched and unsaturated, namely each can have one or more unsaturated parts (double bonds) and one or more substituents along their hydrocarbon chain. Non-limiting example of such fatty acid residues are butyric acid residue (4 carbons), γ-aminobutyric acid residue and a-aminobutyric acid residue, hexanoic acid residue (6 carbons), caprylic acid residue (8 carbons), decanoic acid residue (10 carbons), 5-dodecenoic acid residue, dodec-7-enoic acid residue, lauric acid residue (12 carbons), tetradecanoic acid residue (14 carbons), myristoleic acid residue, tetradec-5-enoic acid residue, tetradec-9-enoic acid residue, palmitic acid residue (16 carbons), hexadec-7-enoic acid residue, hexadec-9-enoic acid residue, palmitoleic acid ((Z)-9-hexadecenoic acid, which is a monounsaturated fatty acid) residue and oleic acid ((Z)-9-octadecanoic acid, which is a monounsaturated fatty acid) residue.

In some embodiments, the fatty acid residue has an amine on the distal carbon of the hydrocarbon chain (with respect to the carboxylic acid group). Such a fatty acid residue is referred to herein as a ω-amino fatty acid residue. Again here the hydrocarbon chain of the ω-amino fatty acid residue may have from 4 to 30 carbon atoms and be saturated or unsaturated and branched or unbranched.

The term “ω-amino-fatty acid” refers to linear amino fatty acids which have an amino group at the end-carbon thereof. Exemplary ω-amino-fatty acids include, without limitation, 4-amino-butyric acid, 6-amino-caproic acid, 8-amino-caprylic acid, 10-amino-capric acid (10-amino-decanoic acid), 12-amino-lauric acid (12-amino-dodecanoic acid), 14-amino-myristic acid (14-amino-tetradecanoic acid), 14-amino-myristoleic acid, 16-amino-palmitic acid (16-amino-hexadecanoic acid), 18-amino-stearic acid, 18-amino-oleic acid, 16-amino-palmitoleic acid, 18-amino-linoleic acid, 18-amino-linolenic acid and 20-amino-arachidonic acid.

In some embodiments of the invention, each of the components in the polymer (monomers), as described herein, is linked to the other by a peptide bond.

The terms “peptide bond” and “amide bond” as used herein refer to an amide group, namely, a —(C═O)NH— group, which is typically formed by nucleophilic addition-elimination reaction between a carboxylic group and an amine group, as these terms are defined herein.

However, the polymers described herein may have other bonds linking the various components in the polymeric structure. Such non-peptidic bonds may render the polymer more stable while in a body or more capable of penetrating into cells. Thus, peptide bonds (—(C═O)NH—) within the polymer may be replaced, for example, by N-methylated amide bonds (—(C═O)NCH₃—), ester bonds (—C(R)H—C(═O)—O—C(R)—N—), ketomethylen bonds (—C(═O)CH₂—), aza bonds (—NH—N(R)—C(═O)—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—(C═—)—), peptide derivatives (—N(R)—CH₂—C(═O)—), wherein R is the “normal” side chain, naturally presented on the carbon atom. These modifications can occur at any of the bonds along the polymer chain and even several (2-3) at the same time.

In some embodiments, all of the bonds in the polymer, linking the various residues to each other, are peptide bonds. For example, in one embodiment, the polymer is made of an amino acid residue linked by a peptide bond to an ω-amino fatty acid residue which in turn is linked to a second amino acid residue by another peptide bond. In another example, the polymer of the previous example is elongated by a second ω-amino fatty acid residue or a fatty acid residue which is linked to any one of the N- or C-termini by a peptide bond, etcetera.

Unless stated otherwise, the use of the terms “polymer” and “polymers” herein refers to both the cyclic and/or the linear form thereof.

The term “linear” as used herein in the context of the polymers, refers to a non-cyclic polymer, i.e., a polymer which have two termini and its backbone or amino-acid side-chains do not form a closed ring.

The term “cyclic” as used herein in the context of the polymer, refers to a polymer that comprises an intramolecular covalent bond between two non-adjacent residues (monomers) therein, forming a cyclic polymer ring.

In the context of the present embodiments the polymer comprises residues of amino acids and hydrophobic moieties which constitute the monomers of the polymer. The term residue is meant to encompass other chemical moieties which form a part of the polymer, and which do not fall under the definition of amino acid or hydrophobic moiety, as these are defined herein. For example, the cyclic polymer may be “closed” or cyclized by means of a multifunctional or bifunctional moiety that will form a part of the cyclic polymer once it is cyclized.

According to some embodiments with respect to the cyclic polymer, the polymer includes at least one residue that has a functional group, which is referred to herein as the first functional group, and at least one residue that has a second functional group, whereas the first and second functional groups are covalently linked therebetween, thereby forming a cyclic polymer.

As used herein, the phrase “functional group” describes a chemical group that is capable of undergoing a chemical reaction that typically leads to a bond formation. According to some embodiments, the bond is a covalent bond. Chemical reactions that lead to a bond formation include, for example, nucleophilic and electrophilic substitutions, nucleophilic and electrophilic addition reactions, addition-elimination reactions, cycloaddition reactions, rearrangement reactions and any other known organic reactions that involve a functional group.

The first and second functional groups may form a part of an amino acid residue and/or a hydrophobic moiety residue in the polymer, or any other element in the polymer which does not fall under the definition of amino acid or hydrophobic moiety, such as, for example, a linking moiety. The first and second functional groups are selected such that they are capable of forming a covalent bond therebetween or therefrom. For example, either the first or the second functional group can be a binding pair of an amine and a carboxyl which form an amide (peptide bond), a hydroxyl and a carboxyl which form an ester, or a an amine and an aldehyde which form an imine (Schiff base).

According to some embodiments, the first functional group is an amine group and the second functional group is a carboxyl group. Alternatively, the first functional group is a carboxyl group and the second functional group is an amine group. Therefore the first functional group and the second functional group can form a peptide bond therebetween.

The amine group, in the context of the first and/or second functional group, can originate from an N-alpha amine of an amino acid residue, or from an amine on the side-chain of an amino acid residue, such as found for example, in lysine and ornithine. Alternatively, the amine can stem from a hydrophobic moiety residue, such as, for example, an amino-fatty acid. Similarly, the carboxyl group, in the context of the first and/or second functional group, can originate from a C-alpha carboxyl of an amino acid residue, or from a carboxyl on the side-chain of an amino acid residue, such as found for example, in aspartic acid and glutamic acid. Alternatively, the amine can stem from a hydrophobic moiety residue, such as, for example, an amino-fatty acid. Similarly, the carboxyl group can stem from a hydrophobic moiety residue, such as, for example, any fatty acid.

Preferably, the one of the first or second functional groups is an amine on a hydrophobic moiety residue, and the other functional group is a carboxyl on an amino acid residue.

As used herein, the term “amine” describes a —NR′R″ group where each of R′ and R″ is independently hydrogen, alkyl, cycloalkyl, heteroalicyclic, aryl or heteroaryl, as these terms are defined herein.

As used herein, the term “alkyl” describes an aliphatic hydrocarbon including straight chain and branched chain groups. Preferably, the alkyl group has 1 to 20 carbon atoms, and more preferably 1-10 carbon atoms. Whenever a numerical range; e.g., “1-10”, is stated herein, it implies that the group, in this case the alkyl group, may contain 1 carbon atom, 2 carbon atoms, 3 carbon atoms, etc., up to and including 10 carbon atoms. The alkyl can be substituted or unsubstituted. When substituted, the substituent can be, for example, an alkyl, an alkenyl, an alkynyl, a cycloalkyl, an aryl, a heteroaryl, a halide, a hydroxy, an alkoxy and a hydroxyalkyl as these terms are defined hereinbelow. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “carboxyl”, as used herein, refers to a —C(═O)—O—R′, where R′ is as defined herein. When R′ is hydrogen the carboxyl group is referred to as a carboxylic acid, and when R′ is an alkyl, the carboxyl group is referred to as an ester.

The term “amide” describes a —NR′—C(═O)— group, a —NR′—C(═O)—R″ group or a —C(═O)—NR′R″ group, wherein R′ is as defined herein and R″ is as defined for R′. An amide is used herein interchangeably with peptide bond.

The term “hydroxyl”, as used herein, refers to an —OH group.

As used herein, the term “aldehyde” refers to a —C(═O)H group.

The term “imine”, which is also referred to in the art interchangeably as “Schiff-base”, describes a —N═CR′— group, with R′ as defined herein. As is well known in the art, Schiff bases are typically formed by reacting an aldehyde and an amine-containing moiety such as amine, hydrazine, hydrazide and the like, as these terms are defined herein.

The polymer as described herein, may have two or more hydrophobic moiety residues as defined hereinabove, whereby at least one is linked to one amino acid at one end and to another amino acid residue at another end, and another may elongate the polymeric chain by being linked to either one of the termini thereof, for example to the N-alpha of a terminal amino acid residue and/or the C-alpha of a terminal amino acid residue. Optionally, a second hydrophobic moiety may be linked to a side-chain of an amino acid residue in the polymer.

The polymer, according to some embodiments, includes from 2 to 50 positively charged amino acid residues. According to other embodiments the polymer includes from 2 to 8 positively charged amino acid residues.

The polymer, according to some embodiments, includes from 1 to 50 hydrophobic moiety residues. Alternatively, the polymer comprises from 1 to 12 hydrophobic moiety residues, or from 1 to 8 hydrophobic moiety residues or from 1 to 6 hydrophobic moiety residues.

The linear polymers described herein can be represented collectively by the following general Formula I:

X-W₀-[A₁-Z₁-D₁]-W₁-[A₂-Z₂-D₂]-W₂- . . . [An-Zn-Dn]-Wn-Y Formula I

wherein:

n is an integer from 2 to 50, preferably from 2 to 12 and more preferably from 2 to 8;

A₁, A₂, . . . , An are each independently a positively charge amino acid residue as discussed hereinabove, such as histidine residues, lysine residues, ornithine residues and arginine residues. In some embodiments all of the positively charged amino acid residues A₁, A₂, . . . , An are lysine residues;

D₁, D₂, . . . , Dn are each independently a hydrophobic moiety residue, as defined and discussed hereinabove, or absent, provided that at least one such hydrophobic moiety residue exists in the polymer. In some embodiments the hydrophobic moiety residues are all ω-amino-fatty acid residues;

Linking moieties connecting each monomer of the polymer, denoted Z₁, Z₂, . . . , Zn and W₀, W₁, W₂, . . . , Wn, each of which independently linking an amino acid residue and a hydrophobic moiety residue or absent. In some embodiments at least two of the linking moieties are a peptide bond and in other embodiments all the linking moieties are peptide bonds;

The fringes of the polymer, denoted X and Y, may each independently be hydrogen, an amine, an amide, an amino acid residue, a hydrophobic moiety residue, an ω-amino-fatty acid residue, a fatty acid residue or absent.

Exemplary linear polymers, as described herein, are those having the structures presented here in below:

which is also referred to herein as C₁₂K(KNC₁₀K)₃NH₂ (SEQ ID NO: 10), or as C₁₂K-3β₁₀;
which is also referred to herein as C₁₂₍₅₋ₑₙₑ₎KKNC₁₂KNH₂ (SEQ ID NO: 2), or as C_{12(107 7)}K-β₁₂; and
which is also referred to herein as C₁₂K(NC₈K)₇NH₂ (SEQ ID NO: 4), or as C₁₂K-7α₈.

Other exemplary linear polymers are presented in U.S. Patent Application Nos. 20070032428, 11/234,183 and 11/500,461 and WO 2006/035431, WO 2008/072242 and WO 2008/132738.

The cyclic polymers described herein can be represented collectively by the following general Formula II:

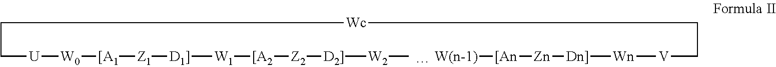

wherein:

n is an integer from 2 to 50, preferably from 2 to 12 and more preferably from 2 to 8;

A₁, A₂, . . . , An are each independently a positively charged amino acid residue, such as histidine residues, lysine residues, ornithine residues and arginine residues, and in some embodiments all the positively charged amino acid residues are lysine residues;

D₁, D₂, . . . , Dn are each independently a hydrophobic moiety residue, as defined and discussed hereinabove, or absent, provided that at least one such hydrophobic moiety residue exists and it is an ω-amino-fatty acid residue;

Connecting each monomer of the residue are linking moieties, denoted Z₁, Z₂, . . . , Zn and W₁, W₂, . . . , Wn−1, each of which independently linking an amino acid residue and a hydrophobic moiety residue or absent.

U is selected from the group consisting of the first functional group, as defined hereinabove, an amino acid residue having that first functional group, a hydrophobic moiety residue having that first functional group, and a linking moiety having that first functional group, or absent.

Similarly, V is selected from the group consisting of the second functional group, an amino acid residue having that second functional group, a hydrophobic moiety residue having that second functional group, and a linking moiety having that second functional group, or absent.

The linking moiety W₀ is linking any one of A₁, Z₁ and D₁ to U, or absent, and the linking moiety Wn is linking any one of An, Zn and Dn to V, or absent;

Wc is a cyclizing moiety.

The moieties which close the polymer into a cyclic polymer, denoted U and V, may each independently be absent or be an amino acid residue or a hydrophobic moiety residue, provided they each has a functional group, referred to hereinabove as the first and second functional groups, which can form a covalent bond therebetween. Thus, such amino acid residues and/or hydrophobic moiety residues can form together a unique linking moiety denoted herein as Wc, which is referred to herein as the cyclizing moiety.

As used herein, the phrase “linking moiety” describes a chemical moiety, group or a bond, as defined herein, which links between two residues or monomers. The linking moiety can thus be, for example, formed upon reacting two functional groups; each forms a part of another monomer or residue, thus linking the two monomers or residues. For example, an amine group on one monomer can form a peptide bond with a carboxyl group on another monomer and the resulting moiety is a peptide bond linking moiety.

Preferably, at least one of the linking moieties in the polymers presented herein is a peptide bond, and most preferable all the linking moieties are peptide bonds.

The phrase “cyclizing moiety”, denoted Wc in Formula II, refers to a chemical moiety which is formed when two residues in Formula II are linked therebetween, thereby forming the cyclic polymer. The cyclizing moiety may be, for example, a bond which is formed between two functional groups, such as, for a non-limiting example, an amide (peptide bond), a carboxylate (ester), a carbamate, an ether and the likes.

The two functional groups which form Wc, can stem from U and V, W₀ and Wn, or A₁, Z₁ and D₁ and An, Zn or Dn, or any combination thereof. Alternatively, the cyclizing moiety may comprise a residue of a multifunctional (as at least bifunctional) moiety which forms bonds with functional groups on U and V, W₀ and Wn, or A₁, Z₁ and D₁ and An, Zn or Dn, such as, for a non-limiting example, p-aminobenzoic acid or ethyleneglycol.

Preferably the cyclizing moiety, denoted Wc, is a peptide bond which is formed from an amine group on either U of V, and a carboxyl on either V or U.

Hence, for better clarity, the phrase “cyclic polymer” as used herein in the context of the polymer, refers to a polymer that comprises an intramolecular covalent bond which forms a part of a cyclizing moiety. The cyclizing moiety is positioned between two non-adjacent residues therein, forming a cyclic polymer ring that comprises at least two amino acid residues, at least one hydrophobic moiety residue, a cyclizing moiety and optionally further comprise a plurality of linking moieties and other residues. The cyclizing moiety may connect backbone to any two residues in the polymer via backbone atoms, side-chain atoms or a combination thereof.

Preferred cyclic polymers are polymers in which n is an integer from 2 to 5, the amino acid residues are all lysine residues, and the hydrophobic moiety residues are all 12-amino-lauric acid residues.

Exemplary cyclic polymers, as described herein, are those having the structures presented hereinbelow:

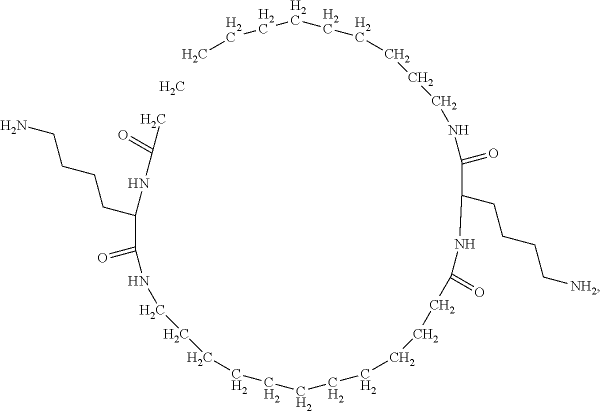

which is also referred to herein as Cyclic-(NC₁₂K)₂; and

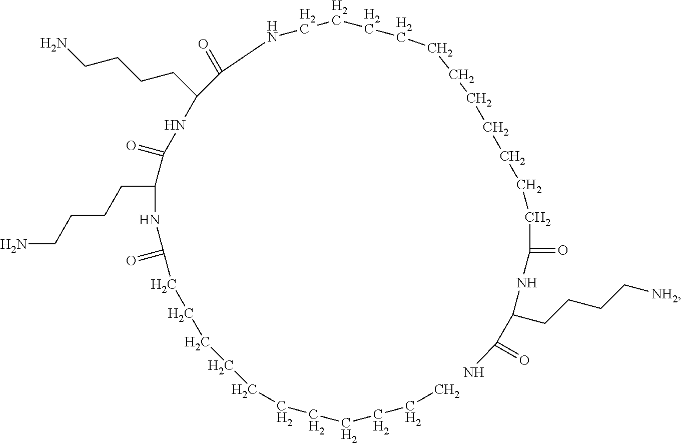 [SMILES: CCCCCCCCCCCC(=O)NC(CCCCN)C(=O)NC(CCCCN)C(=O)NCCCCCCCCCCCC(=O)NC(CCCCN)C(N)=O]

which is also referred to herein as Cyclic-NC₁₂KKNC₁₂K.

As discussed above, one or more of the hydrophobic moiety residues may be attached to a side chain of one or more of the amino acid residues of the polymer, i.e., act as a branch of the main linear or cyclic polymer.

The antimicrobial re-sensitizing polymers described herein can be readily synthesized as demonstrated for structurally similar polymers in U.S. Patent Application Nos. 20070032428, 11/234,183 and 11/500,461 and WO 2006/035431, WO 2008/072242 and WO 2008/132738. For example, polymers in which the linking moieties are peptide bonds, and hence resemble natural and synthetic peptides in this respect, can be prepared by classical methods known in the art for peptide syntheses. Such methods include, for example, standard solid phase techniques. The standard methods include exclusive solid phase synthesis, partial solid phase synthesis methods, fragment condensation, classical solution synthesis, and even by recombinant DNA technology. See, e.g., Merrifield, J. Am. Chem. Soc., 85:2149 (1963), incorporated herein by reference. Solid phase peptide synthesis procedures are well known in the art and further described by John Morrow Stewart and Janis Dillaha Young, Solid Phase Peptide Syntheses (2nd Ed., Pierce Chemical Company, 1984).

The antimicrobial re-sensitizing polymers described herein can be purified, for example, by preparative high performance liquid chromatography [Creighton T. (1983) Proteins, structures and molecular principles. WH Freeman and Co. N.Y.].

In a search for highly effective antimicrobial re-sensitizing polymers, the present inventors have prepared and successfully practiced novel antimicrobial polymers.

Hence, according to another aspect of embodiments of the invention, there are provided novel polymers, each including a plurality of positively charged amino acid residues and more than one ω-amino-fatty acid residue, as described herein, and further having an antimicrobial re-sensitizing activity.

Exemplary such polymers include the following: C₁₂₍₅₋ₑₙₑ₎KKNC₁₂KNH₂ (SEQ ID NO: 2), C₁₄₍₉₋ₑₙₑ₎KKNC₁₂KNH₂ (SEQ ID NO: 5), C₁₆₍₉₋ₑₙₑ₎KKNC₁₂KNH₂ (SEQ ID NO: 6) and C₁₂K(KNC₁₀K)₃NH₂ (SEQ ID NO: 10).

Further according to embodiments of the invention, there are provided pharmaceutical compositions comprising these novel polymers and uses thereof as medicaments.

As described herein, these novel polymers can be advantageously used as antimicrobial re-sensitizing polymers, for treating medical conditions associate with pathogenic microorganism in subjects diagnosed as having the medical condition which were treated with an antimicrobial agent and following an emergence of resistance to the anti-microbial agent, as described herein.

Further provided are processes of preparing these novel polymers, as described herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Experimental Methods

Chemical Syntheses and Analysis of Antimicrobial Re-Sensitizing Polymers:

The polymers were produced by the solid phase method following methodologies disclosed in U.S. Patent Application Nos. 20070032428, 11/234,183 and 11/500,461 and WO 2006/035431, WO 2008/072242 and WO 2008/132738, which are all incorporated by reference as if fully set forth herein.

Briefly, the polymers were synthesized while applying the Fmoc active ester chemistry on a fully automated, programmable peptide synthesizer (Applied Biosystems 433A). After cleavage from the resin, the crude product was extracted with 30% acetonitrile in water and purified by RP-HPLC (Alliance Waters), so as to obtain a chromatographic homogeneity higher than 95%. HPLC runs were typically performed on C₁₈ columns (Vydac, 250 mm×4.6 or 10 mm) using a linear gradient of acetonitrile in water (1% per minute), both solvents containing 0.1% trifluoroacetic acid. The purified polymers were subjected to mass spectrometry (ZQ Waters) and NMR analyses to confirm their composition and stored as a lyophilized powder at −20° C. Prior to being tested, fresh solutions were prepared in water, vortexed, sonicated, centrifuged and then diluted in the appropriate medium.

Non-Polymer Antimicrobial Agents:

In order to demonstrate the re-sensitizing activity of the polymers according to embodiments of the invention, sensitive (susceptible) and resistant bacterial strains were tested for their response to several non-polymer antimicrobial agents, such as oxacillin, piperacillin, penicillin G, ciprofloxacin, erythromycin, tetracycline, gentamicin and methicillin.

Bacterial Strains and Sample Preparation:

Antibacterial activity was determined using various strains of S. aureus, E. coli P. aeruginosa, P. mirabilis and S. maltophila, cultured in LB medium (10 grams/liter trypton, 5 grams/liter yeast extract, 5 grams/liter NaCl, pH 7.4). Bacterial strains include susceptible strains of Staphylococcus aureus, Pseudomonas aeruginosa, Bacillus cereus and Escherichia coli (ATCC, American Type Culture Collection), as well as antibiotic-resistant strains such as oxacillin-resistant Staphylococcus aureus (ORSA) and methicillin-resistant Staphylococcus aureus (MRSA).

Minimal Inhibitory Concentration (MIC) Measurements:

Minimal Inhibition Concentration (MIC) is defined as the lowest drug concentration that induced a 100% inhibition of proliferation at standard growth conditions. MICs were determined by microdilution susceptibility testing in 96-well plates (by Nunc) using inocula of 10⁵ bacteria per ml. Cell populations were evaluated by optical density measurements at 620 nm and were calibrated against a set of standards. Hundred (100) μl of a bacterial suspension were added to 100 μl of culture medium (control) or to 100 μl of culture medium containing various polymer concentrations in 2-fold serial dilutions. Inhibition of proliferation was determined by optical density measurements at 620 nm after an incubation period of 24 hours at 37° C.

Induction of Resistance and its Reversal:

For each tested polymer, the culture displaying one half MIC (based on optical density measurements at 620 nm) was diluted in LB to yield 5×10⁵ CFU/ml (according to a calibration curve) and used again for the subsequent MIC determination. In parallel, MIC evolution was compared concomitantly for each new sub-culture, using bacteria harvested from control wells (wells cultured without antimicrobial agent from the previous sub-culture). The relative MIC was calculated for each experiment from the ratio of MIC obtained for subculture “n” to that obtained for first-time exposure. As can be seen in the Results section below, the mean polymer's MIC (determined from 10 independent experiments performed in duplicates) was 6.2 μM (4.9 and 6.9 μg/ml respectively for C₁₂₍₅₋ₑₙₑ₎KKNC₁₂KNH₂ (SEQ ID NO: 2) and NC₁₂(KNC₁₂K)₂NH₂ (SEQ ID NO: 1)). Oxacillin's MIC was 0.28 μM=0.125 μg/ml (0.125-0.5 μg/ml, as determined by the Clinical and Laboratory Standard Institute, M100-S15).

### Experimental Results

Previous studies, as well as U.S. Patent Application Nos. 20070032428, 11/234,183 and 11/500,461 and WO 2006/035431, have shown that the acyl-lysine antimicrobial polymers exert sequence dependant bacteriostatic and/or bactericidal effects with in-vitro MIC at low micromolar range and in-vivo efficacy at low mg/Kg range.

According to some embodiments of the present invention, the polymers exhibit antimicrobial re-sensitizing activities with respect to other antibiotics using bacterial cultures exposed to sub-MIC polymers concentrations, namely at concentrations wherein the polymers alone are not active. Hence, an effective re-sensitizing amount for the polymers according to embodiments of the invention is lower than their effective therapeutic amount, or MIC.

Exemplary Polymers Library:

Several representative polymers according to the present embodiments, which are substantially comprised of a plurality of fatty acid (acyl) residues, lysine residues and ω-amino-fatty acid residues, also referred to herein and elsewhere as oligo-acyl-lysines or OAKs, were prepared according to the general procedure described in U.S. Patent Application Nos. 20070032428, 11/234,183 and 11/500,461 and WO 2006/035431, WO 2008/072242 and WO 2008/132738, and are presented in Table 3 below.

The polymers in this section can be described using the shorthand denotations described below.

N or NH₂, when present, denotes an amino group, which may be a terminal group such as in a primary amine at the N-terminus of the polymer or a part of an amide at the C-terminus of the polymer, and may be a part of the peptide bond connecting two polymer residues;

The polymer residue NC_{i(y)} denotes an ω-amino-fatty acid residue, and polymer residue C_{(i(y)} denotes a fatty acid residue, whereby i denotes the number of carbon atoms in the aliphatic chain thereof and (y) denotes a double bond along the chain, e.g. for NC₁₂₍₅₋ₑₙₑ₎, i is 12 and (y) is (5-ene) and the residue is of 12-amino-5-dodecenoic acid residue acid, whereby when the denotation (y) is absent, it is meant that the chain is saturated, e.g. C₁₂ denotes a residue of lauric acid;

The polymer residue K(x) denotes a lysine residue, wherein (x) denotes the type of amine group in the amino acid which is used for conjugation with another residue in the polymer, whereby when the denotation (x) is absent, it is meant that conjugation is effected via the N-alpha of the lysine residue and when (x) is (c) it is meant that conjugation is effected via the epsilon amine of the lysine residue;

The polymers presented herein and in U.S. Patent Application Nos. 20070032428, 11/234,183 and 11/500,461 and WO 2006/035431, WO 2008/072242 and WO 2008/132738, can be cyclic polymers, whereby the prefix “Cyclic-” is added to the denotation to mark a cyclic polymer. When cyclic, the polymer's termini form a linking moiety. For example, the linking moiety can be a peptide bond which forms between a terminal amine of an ω-amino-fatty acid residue and a terminal carboxyl of a lysine residue.

These exemplary polymers are referred to in this section according to the following formula:

T[NCᵢK(x)]ⱼG or Cyclic-T[NCᵢK(x)]ⱼG

In this formula, NCᵢ or NC_{i(y)} denotes an ω-amino-fatty acid residue (an exemplary hydrophobic moiety according to the present invention, represented by D₁ . . . Dn in the general formulae I and II described herein); K(x) denotes a lysine residue (an exemplary amino acid residue according to the present invention, denoted as A₁ . . . An in the general Formulae I and II described herein, such that [NCᵢK(x)] denotes a residue of an ω-amino-fatty acid-lysine conjugate (denoted as [A₁-Z₁-D₁] . . . [An-Zn-Dn] in the general Formulae I and II described herein); j denotes the number of the repeating units of a specific conjugate in the polymer (corresponding to n in the general Formulae I and II described herein); and T and G each independently denotes either a hydrogen (no denotation), a lysine residue (denoted K), an amidated lysine residue (denoted KNH₂), an ω-amino-fatty acid residue (denoted NCᵢ or NC_{i(y)}), a fatty acid residue (denoted Cᵢ or C_{i(y)}), an ω-amino-fatty acid-lysine conjugate residue (denoted NCᵢK or NC_{i(y)}K), a fluorenylmethyloxycarbonyl residue (denoted Fmoc), a benzyl residue (denoted Bz), a tert-butylcarbonyl residue (denoted t-Boc or Boc), an amine group (typically forming an amide at the C-terminus and denoted NH₂), and free acid residue (for the C-terminus no denotation), an alcohol residue, and any combination thereof (all corresponding to X and Y in the general Formula I described herein).

Thus, for example, a polymer according to embodiments of the present invention which is referred to herein as C₁₂K(NC₈K)₇NH₂ (SEQ ID NO: 4), corresponds to a polymer having the general Formula I described hereinabove, wherein: X is a residue of a conjugate of a fatty acid having 12 carbon atoms (lauric acid) and lysine; n is 6; A₁ . . . A₆ are each a lysine residue; D₁ . . . D₇ are all residues of an ω-amino-fatty acid having 8 carbon atoms (8-amino-caprylic acid); Z₁ . . . Z₇ and W₀-W₇ are all peptide bonds; and Y is an amine. For clarity, the chemical structure of C₁₂K(NC₈K)₇NH₂ (SEQ ID NO: 4) is presented in Scheme 1 below:

For another example, a polymer according to the present embodiments which is referred to herein as C₁₂K(ϵ)NC₁₂K(ϵ)NH₂, corresponds to a polymer having the general Formula I described hereinabove, wherein: X is a residue of a conjugate of an ω-amino-fatty acid having 12 carbon atoms (12-amino-lauric acid) and lysine; n is 61 hence not denoted; A₁ . . . A₆ A₂ are each a lysine residue, both conjugated via the epsilon amine hence denoted K(ϵ); D₁ . . . D₇ are all is a residues of an ω-amino-fatty acid having 12 carbon atoms (12-amino-lauric acid); Z₁ . . . Z₇ Z₂ and W₀-W₇₁ are all peptide bonds; and Y is an amine that forms a part of the amidated terminal lysine residue. For clarity, the chemical structure of C₁₂K(ϵ)NC₁₂K(ϵ)NH₂ is presented in Scheme 2 below:

For another example, a polymer according to the present invention which is referred to herein as C₁₂₍₅₋ₑₙₑ₎KKNC₁₂KNH₂ (SEQ ID NO: 2), corresponds to a polymer having the general Formula I described hereinabove, wherein: X is a 5-dodecenoic acid residue; D₁ and D₁ are absent, D₃ is a residue of an ω-amino-fatty acid having 12 carbon atoms (12-amino-lauric acid); Z₁ . . . Z₂ and W₁ are all peptide bonds; and Y is an amine that forms a part of the amidated terminal lysine residue. For clarity, the chemical structure of C₁₂₍₅₋ₑₙₑ₎KKNC₁₂KNH₂ (SEQ ID NO: 2) is presented in Scheme 3 below:

Table 3 below presents the exemplary polymers comprising a plurality of, lysine residues and ω-amino-fatty acid and fatty acid (acyl) residues, referred to herein interchangeably (particularly in the Figures) as oligo-acyl-lysines or OAKs, according to some embodiments of the present invention, which were tested for their antimicrobial re-sensitizing capacity.

TABLE 3

| SEQ ID | | Alternative | |
|---|---|---|---|
| NO: | Polymer sequence | denotation | Prior reference |
| 1 | NC₁₂(KNC₁₂K)₂NH₂ | NC₁₂-2β₁₂ | U.S. Pat. App. |
| | | | 2007/0032428 |
| 2 | C₁₂₍₅₋ₑₙₑ₎KKNC₁₂KNH₂ | C_{12(ω7)}K-β₁₂ | None |
| 3 | C₁₂K(NC₈K)₅NH₂ | C₁₂K-5α₈ | U.S. Pat. App. |
| | | | 2007/0032428 |
| 4 | C₁₂K(NC₈K)₇NH₂ | C₁₂K-7α₈ | U.S. Pat. App. |
| | | | 2007/0032428 |
| 5 | C₁₄₍₉₋ₑₙₑ₎KKNC₁₂KNH₂ | C_{14(ω5)}K-β₁₂ | None |
| 6 | C₁₆₍₉₋ₑₙₑ₎KKNC₁₂KNH₂ | C_{16(ω7)}K-β₁₂ | None |
| 7 | C₁₂KKNC₁₂KNH₂ | C₁₂K-β₁₂ | U.S. Pat. App. |
| | | | 2007/0032428 |
| 8 | C₁₂K(KNC₁₂K)₂NH₂ | C₁₂K-2β₁₂ | U.S. Pat. App. |
| | | | 2007/0032428 |
| 9 | C₁₂K(KNC₁₂K)₃NH₂ | C₁₂K-3β₁₂ | WO 2008/132738 |
| 10 | C₁₂K(KNC₁₀K)₃NH₂ | C₁₂K-3β₁₀ | None |

Potentiation of Non-Polymer Antimicrobial Agents:

Oxacillin, or oxacillin sodium, also known as Bactocill, is a narrow spectrum β-lactam antibiotic derived from penicillin. Traditionally it has been used to fight Staphylococcus aureus infections. However its use is now limited since the emergence of resistant strains, referred to as oxacillin-resistant Staphylococcus aureus or ORSA. Oxacillin was used to demonstrate the antimicrobial re-sensitizing capacity of the polymers according to the present embodiments.

The fractionary inhibitory (FIC) concentration index for combinations of two antimicrobial agents was calculated as follows: FIC index=FICA+FICB=A/MICA+B/MICB where A and B are the MIC values of agent A and agent B in the combination, MICA and MICB are the MICs of agent A and agent B alone, and FICA and FICB are the FICs of agent A and agent B.

The FIC indexes were interpreted as follows: less than 0.5 indicates a clear re-sensitizing effect; from 0.5 to 4 indicates a marginal or null re-sensitizing effect; and over 4 indicates antagonism.

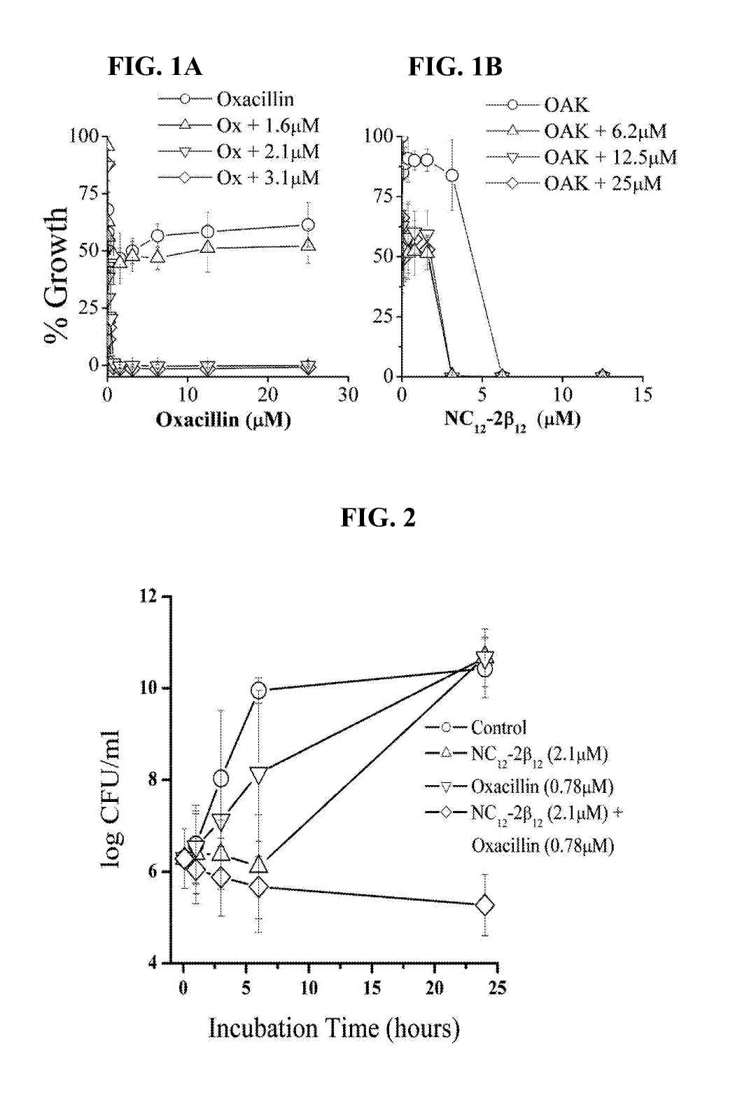

FIGS. 1A-1B present a comparative plot of bacterial growth of methicillin resistant Staphylococcus aureus (MRSA 15903, a clinical isolate) versus antimicrobial agent concentration, demonstrating in FIG. 1A that while oxacillin alone was inactive at least up to 25 μM, the presence of the polymer NC₁₂(KNC₁₂K)₂NH₂ (SEQ ID NO: 1) (also referred to as NC₁₂-2β₁₂) at concentrations well below it MIC value (e.g. ⅓ and ½ MIC, when the MIC is 6.25 μM) have endowed potency to oxacillin, or enhanced the sensitivity of the bacteria thereto (from 576 μM=256□μg/ml the MIC became 0.8 μM=0.34 and 0.4 μM=0.17 μg/ml, respectively) and an optimal re-sensitization concentration of 2.1 μM exhibiting a fractionary inhibitory concentration (FIC) index of 0.34, and further demonstrating in FIG. 1B that in presence of oxacillin there was merely a twofold improvement in the polymer's MIC, indicating that oxacillin did not potentiate the effect of the polymer.

The data used for the plots of FIG. 1A-B were obtained as follows: one hundred (100) μl of a bacterial suspension (10⁵ bacteria per ml) were added to 100 μl of culture medium (control) or to 100 μl of culture medium containing various oxacillin concentrations in 2-fold serial dilutions in presence of the specified sub-MIC polymer concentrations (FIG. 1A). In the opposite experiment (FIG. 1B) the polymer concentrations in serial twofold dilutions were assessed in presence of the specified sub-MIC oxacillin values. Proliferation was determined by optical density measurements at 620 nm after an incubation period of 24 hours at 37° C.

Table 4 presents the individual MIC values of oxacillin and two exemplary polymers, NC₁₂(KNC₁₂K)₂NH₂ (SEQ ID NO: 1) and C₁₂₍₅₋ₑₙₑ₎KKNC₁₂KNH₂ (SEQ ID NO: 2) (also referred to as C_{12(ω7)}K-β₁₂), over various strains of Staphylococcus aureus.

TABLE 4

| | NC₁₂(KNC₁₂K)₂NH₂ | C₁₂₍₅₋ₑₙₑ₎KKNC₁₂KNH₂ | |
|---|---|---|---|
| | (SEQ ID NO: 1) | (SEQ ID NO: 2) | Oxacillin |
| Strain | (MIC in μM) | (MIC in μM) | (μg/ml) |
| MRSA | 6.2 | 3.1-6.2 | 256 |
| 15903 | | | |
| MRSA | 6.2 | 6.2 | 64 |
| 15918 | | | |
| MRSA | 3.1 | 6.2 | 128 |
| 15852 | | | |
| ATCC | 3.1 | 3.1 | 0.17 |
| 25923 | | | |
| ATCC | 3.1 | 6.2 | 0.09 |
| 29213 | | | |

Table 5 presents the fractionary inhibitory concentration (FIC) index against three resistant strains (MRSA) and a susceptible strain (ATCC) of Staphylococcus aureus, calculated for oxacillin activity in presence of the polymers concentration corresponding to ⅓ of the polymer's MIC value.

TABLE 5

| | FIC at ⅓ polymer MIC | |
|---|---|---|
| | NC₁₂(KNC₁₂K)₂NH₂ | C₁₂₍₅₋ₑₙₑ₎KKNC₁₂KNH₂ |
| Strain | (SEQ ID NO: 1) | (SEQ ID NO: 2) |
| MRSA 15903 | 0.30 | 0.35 |
| MRSA 15918 | 0.31 | not determined |
| MRSA 15852 | 0.33 | not determined |
| ATCC 29213 | 0.75 | not determined |

Bactericidal activity is defined as a 3 log₁₀ decrease in CFU/ml from the most active single agent whereas potentiation of one agent by another is defined as a 2 log₁₀ decrease after 24 hours of incubation in presence of the combination of the two agents compared to the most active single agent when the number of surviving organisms in presence of the combination is higher or equal to 2 log₁₀ CFU/ml below the starting inoculum.

FIG. 2 presents comparative plots of the colony-forming unit (CFU) of MRSA 15903 (a clinical isolate) versus incubation time, showing the sub-MIC time-kill curves obtained for oxacillin or NC₁₂(KNC₁₂K)₂NH₂ (SEQ ID NO: 1), as an exemplary polymer, alone and in combination at low individual concentrations, supporting the findings presented hereinabove and in FIGS. 1A-B.

Experimental data was obtained as follows: 100 μl of bacterial suspension (10⁶ bacteria per ml) in culture medium were added to 1 ml of culture medium-containing zero (control) or NC₁₂(KNC₁₂K)₂NH₂ (SEQ ID NO: 1) in sub-MIC concentration (2.1 μM) or a combination of oxacillin (0.78□μM) and NC₁₂(KNC₁₂K)₂NH₂ (SEQ ID NO: 1) (2.1 μM). At the specified time points of incubation (37° C. under shaking), cultures were subjected to serial 10-fold dilutions (up to 10⁶) by adding 50 μl of sample to 450 μl saline (0.85% NaCl). Cell counts were determined using the drop plate method (three 20-μl drops onto LB-agar plates). Plates were incubated at 37° C. and colonies were counted for the calculation after incubation for 24 hours.

As can be seen in FIG. 2, no bactericidal activity was observed for any of the antimicrobial agents alone, however, a reduction of bacterial counts of 4.7 log₁₀ CFU/ml was seen after 24 hours of incubation upon combinations of oxacillin (0.78□μM) and NC₁₂(KNC₁₂K)₂NH₂ (SEQ ID NO: 1) (2.1 μM) compared to the most active single agent. The number of surviving organisms in presence of the combination is 0.76 log₁₀ CFU/ml below the starting inoculum.

Induction of Oxacillin-Resistance and its Reversal by NC₁₂(KNC₁₂K)₂NH₂ (SEQ ID NO: 1):

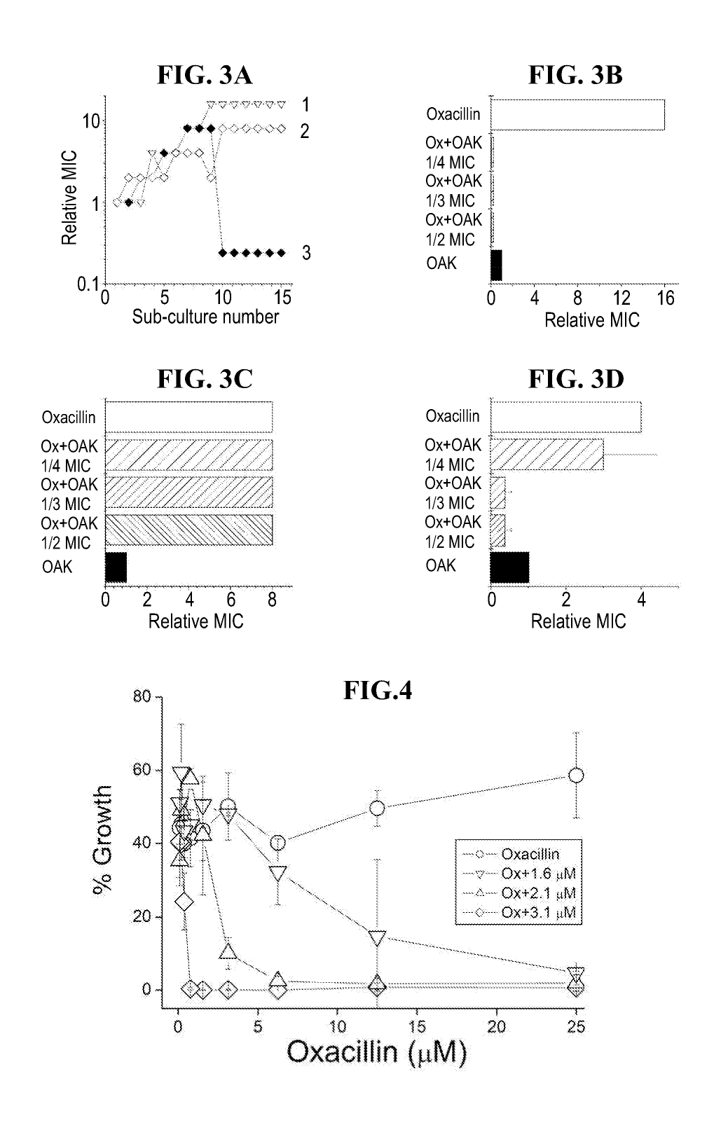

Oxacillin-resistance was induced in S. aureus by culturing the bacteria for up to 15 consecutive generations in the presence of oxacillin. The re-sensitizing effect of NC₁₂(KNC₁₂K)₂NH₂ (SEQ ID NO: 1) as an exemplary re-sensitizing polymer was demonstrated by exposing these oxacillin-resistant cultures to combinations of oxacillin and NC₁₂(KNC₁₂K)₂NH₂ (SEQ ID NO: 1) at sub-MIC values of the polymer. The relative MIC was determined as the normalized ratio of the MIC obtained for a given subculture to the MIC of the control. In addition, the 15th subculture from each experiment was tested against oxacillin or polymer alone and against combinations of oxacillin with the polymer (MIC=6.2 μM=6.9 μg/ml), and the results are presented in FIGS. 3A-3D.

FIGS. 3A-D present the results of experimental induction of oxacillin-resistance in S. aureus and re-sensitization of the bacteria to oxacillin, wherein FIG. 3A shows the emergence of resistance of S. aureus (ATCC 29213, an oxacillin-sensitive strain) when exposed to oxacillin alone (line 1 marked by white triangles in FIG. 3A) or to mixtures of oxacillin and sub-MIC concentrations of NC₁₂(KNC₁₂K)₂NH₂ (SEQ ID NO: 1) (¼ and ⅓ MIC, respectively, lines 2 and 3 marked by white and black diamonds respectively in FIG. 3A), and wherein FIGS. 3B-3D represent attempts to re-sensitize the oxacillin-resistant bacteria shown in FIG. 3A by exposing bacteria from the 15th subcultures (culture shown in line 1 in FIG. 3A corresponds to FIG. 3B, culture shown in line 2 in FIG. 3A corresponds to FIG. 3C and culture shown in line 3 in FIG. 3A corresponds to FIG. 3D) to oxacillin or polymer alone or to mixtures of oxacillin and sub-MIC concentrations of the polymer

The data presented in FIGS. 3A-3D were obtained from at least two independent experiments performed in duplicates, as follows.

For FIG. 3A: For each experiment (oxacillin alone or combinations of oxacillin and NC₁₂(KNC₁₂K)₂NH₂ (SEQ ID NO: 1) at sub-MIC values of the polymer) the culture displaying one half MIC (based on optical density measurements at 620 nm) was diluted in LB to yield 5×10⁵ CFU/ml (according to a calibration curve) and used again for the subsequent MIC determination. In parallel, MIC evolution was compared concomitantly for each new sub-culture, using bacteria harvested from control wells (wells cultured without antimicrobial agent from the previous sub-culture). The relative MIC was calculated for each experiment from the ratio of MIC obtained for subculture “n” to that obtained for first-time exposure.

For FIGS. 3B-D: For each tested treatment (oxacillin alone or a combinations of oxacillin and NC₁₂(KNC₁₂K)₂NH₂ (SEQ ID NO: 1) at sub-MIC values of the polymer) the culture displaying one half MIC in the last subculture (n=15) was diluted in LB to yield 5×10⁵ CFU/ml (according to a calibration curve) and used for MIC determination with each one of the treatments. MICs were determined by microdilution susceptibility testing in 96-well plates using inocula of 10⁵ bacteria per ml. Cell populations were evaluated by optical density measurements at 600 nm and were calibrated against a set of standards.

Table 6 presents the MIC values of various antibiotics alone and in presence of three sub-inhibitory concentrations of NC₁₂(KNC₁₂K)₂NH₂ (SEQ ID NO: 1) against MRSA 15903, and illustrates the fact that NC₁₂(KNC₁₂K)₂NH₂ (SEQ ID NO: 1) was able to potentiate the effect of various antibiotics known to act by distinct mechanisms, namely oxacillin, piperacillin and penicillin G, which are all are β-lactam compounds that inhibit cell wall synthesis; ciprofloxacin inhibits DNA-gyrase activity; erythromycin, tetracycline and gentamicin inhibit ribosomal synthesis of proteins.

TABLE 6

| | MIC (μg/ml) | | | |
|---|---|---|---|---|
| | | | | +½ |
| | | +¼ polymer | +⅓ polymer | polymer |
| | | MIC | MIC | MIC |
| Antibiotics | Alone | (1.6 μM) | (2.1 μM) | (3.1 μM) |
| Oxacillin | 256 | 256 | 0.34 | 0.17 |
| Piperacillin | 256 | 64 | 4 | 1 |
| Penicillin G | 64 | 8 | 2 | 1 |
| Ciprofloxacin | 64 | 32 | 16 | 1 |
| Erythromycin | 0.5 | 1 | 0.125 | 0.016 |
| Tetracycline | 0.5 | 0.5 | 0.25 | 0.004 |
| Gentamicin | 256 | 128 | 128 | 128 |

Potentiation of Oxacillin by C₁₂₍₅₋ₑₙₑ₎KKNC₁₂KNH₂ (SEQ ID NO: 2) against MRSA:

The experimental results presented below illustrate the fact that another exemplary polymer having a different sequence and composition, namely C₁₂₍₅₋ₑₙₑ₎KKNC₁₂KNH₂ (SEQ ID NO: 2) (also referred to as C_{12(ω7)}K-β₁₂ shown below, versus NC₁₂(KNC₁₂K)₂NH₂ (SEQ ID NO: 1) shown above, was able to potentiate the effect of various antibiotics known to act by distinct mechanisms.

Staphylococcal inhibition by oxacillin in combinations with an exemplary re-sensitizing polymer, C₁₂₍₅₋ₑₙₑ₎KKNC₁₂KNH₂ (SEQ ID NO: 2), was demonstrated as shown in FIG. 4 and summarized in Table 7 below. The MIC values against different strains of Staphylococcus aureus and the FIC index are presented hereinabove in Tables 4 and 5, respectively.

FIG. 4 presents comparative plots of bacterial growth of Staphylococcus aureus MRSA 15903 versus concentration of oxacillin with or without potentiation by C₁₂₍₅₋ₑₙₑ₎KKNC₁₂KNH₂ (SEQ ID NO: 2), demonstrating that the presence of the polymer at concentrations well below the MIC value, namely ¼ MIC, endows potency to oxacillin at an optimal polymer concentration of 2.1 μM corresponding to a FIC index of 0.35.

Experimental data for FIG. 4 were obtained as follows. One hundred (100) μl of a bacterial suspension (10⁵ bacteria per ml) were added to 100 μl of culture medium (control) or to 100 μl of culture medium containing various oxacillin concentrations in 2-fold serial dilutions in presence of the specified sub-MIC polymer concentrations. Proliferation was determined by optical density measurements at 620 nm after an incubation period of 24 hours at 37° C.

Table 7 presents the results measured for C₁₂₍₅₋ₑₙₑ₎KKNC₁₂KNH₂ (SEQ ID NO: 2) at three sub-MIC concentrations (MIC=6.2 μM) which demonstrate its capacity to potentiate the effect of various antibiotics. In addition, Table 7 shows that the polymer can potentiate the effect of other polymers such as the exemplary C₁₂K(NC₈K)₅NH₂ (SEQ ID NO: 3) (also referred to as C₁₂K-5α₈) and C₁₂K(NC₈K)₇NH₂ (SEQ ID NO: 4) (also referred to as C₁₂K-7α₈).

TABLE 7

| | MIC (μg/ml) | | | |
|---|---|---|---|---|
| | S. aureus CI-15903 | | | |
| | | +polymer | +polymer | +polymer |
| Antibiotics | Alone | (½ MIC) | (⅓ MIC) | (¼ MIC) |
| Oxacillin | 256 | 0.25 | 4 | 64 |
| Piperacillin | 256 | 0.25 | 4 | 32 |
| Penicillin G | 64 | 0.25 | 4 | 32 |
| Ciprofloxacin | 64 | 0.25 | 4 | 32 |
| Erythromycin | 0.5 | 0.1 | 0.1 | 0.5 |
| Tetracycline | 0.5 | 0.06 | 0.1 | 0.5 |
| Gentamicin | 256 | 128 | 128 | 256 |
| C₁₂K-5α₈ | >50 | 12.5 | 12.5 | 50 |
| C₁₂K-7α₈ | 50 | 6.2 | 12.5 | 25 |

Re-Sensitization of Oxacillin-Resistant Bacteria by C₁₂₍₅₋ₑₙₑ₎KKNC₁₂KNH₂ (SEQ ID NO: 2):

Emergence of resistance to oxacillin or to an antimicrobial polymer alone, and to mixtures of oxacillin and sub-MIC concentration of the exemplary polymer C₁₂₍₅₋ₑₙₑ₎KKNC₁₂KNH₂ (SEQ ID NO: 2), was assessed against the oxacillin-sensitive S. aureus ATCC 29213 strain by measuring MIC value daily for ten consecutive sub-cultures.

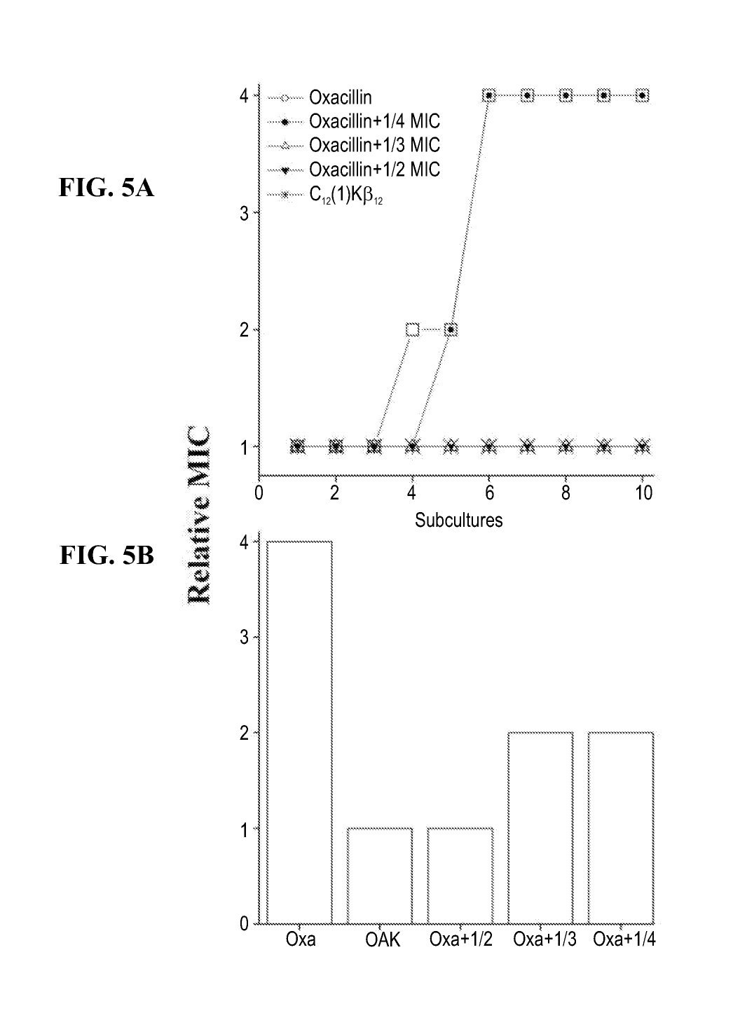

FIGS. 5A-B present the results of the experimental induction of oxacillin-resistance in Staphylococcus aureus (ATCC 29213, an oxacillin-sensitive strain) and re-sensitization of the resistant bacteria, wherein FIG. 5A is a comparative plot of relative MIC of oxacillin versus the bacteria generation, showing that the relative MIC of oxacillin alone or in presence of the lowest re-sensitizing polymer concentration (¼ MIC=1.6 μM) has increased by 4 folds, reflecting emergence of resistance, unlike the effect recorded for the polymer alone or oxacillin combined with third or half the MIC of the exemplary re-sensitizing polymer C₁₂₍₅₋ₑₙₑ₎KKNC₁₂KNH₂ (SEQ ID NO: 2), and wherein FIG. 5B is a bar graph showing the relative MIC obtained for the oxacillin-sensitive S. aureus ATCC 29213 strain when the now-resistant strain (after 10 subcultures in presence of oxacillin) was exposed again to either oxacillin or the re-sensitizing polymer alone or to mixtures of oxacillin and sub-MIC concentrations of the polymer, demonstrating that the relative MIC of oxacillin remained 4, however using the polymer alone or mixtures of oxacillin and sub-MIC polymer concentrations decreased the relative MIC and caused re-sensitization of the bacteria.

Experimental data for FIGS. 5A-5B were obtained as follows.

For FIG. 5A) For each experiment (oxacillin alone or combinations of oxacillin and C₁₂₍₅₋ₑₙₑ₎KKNC₁₂KNH₂ (SEQ ID NO: 2) at sub-MIC values of the polymer) the culture displaying one half MIC (based on optical density measurements at 620 nm) was diluted in LB to yield 5×10⁵ CFU/ml (according to a calibration curve) and used again for the subsequent MIC determination. In parallel, MIC evolution was compared concomitantly for each new generation, using bacteria harvested from control wells (wells cultured without antimicrobial agent from the previous generation). The relative MIC was calculated for each experiment from the ratio of MIC obtained for subculture “n” to that obtained for first-time exposure. B) The 10^{th} oxacillin subculture from the experiment in panel A was diluted in LB to yield 5×10⁵ CFU/ml (according to a calibration curve) and used for MIC determination with each one of the treatments. MICs were determined by microdilution susceptibility testing in 96-well plates. Cell populations were evaluated by optical density measurements at 600 nm and were calibrated against a set of standards.

Potentiation of Non-Polymer Antimicrobial Agents by Various Polymers:

The results presented below demonstrate the re-sensitizing activity of some exemplary antimicrobial re-sensitizing polymers, C₁₂₍₅₋ₑₙₑ₎KKNC₁₂KNH₂ (SEQ ID NO: 2) (Table 8 hereinbelow), C₁₄₍₉₋ₑₙₑ₎KKNC₁₂KNH₂ (SEQ ID NO: 5) (also referred to as C_{14(ω5)}K-β₁₂, Table 9 hereinbelow), C₁₆₍₉₋ₑₙₑ₎KKNC₁₂KNH₂ (SEQ ID NO: 6) (also referred to as C_{16(ω7)}K-β₁₂, Table 10 hereinbelow), C₁₂KKNC₁₂KNH₂ (SEQ ID NO: 7) (also referred to as C₁₂K-β₁₂, Table 11 hereinbelow), C₁₂K(KNC₁₂K)₂NH₂ (SEQ ID NO: 8) (also referred to as C₁₂K-2β₁₂, Table 12 hereinbelow) and C₁₂K(KNC₁₂K)₃NH₂ (SEQ ID NO: 9) (also referred to as C₁₂K-3β₁₂, Table 13 hereinbelow), according to embodiments of the present invention, when acting together with a series of classical antibiotic agents, such as oxacillin, piperacillin, penicillin G, ciprofloxacin, erythromycin, tetracycline, gentamicin and methicillin, against sensitive and resistant bacterial strains.

Table 8 below presents the MIC values of antibiotics in presence or absence of C₁₂₍₅₋ₑₙₑ₎KKNC₁₂KNH₂ (SEQ ID NO: 2) at sub-MIC levels of the polymer, as measured for the S. aureus strains 43300 (ATCC), 15819 (clinical isolate), 15877 (clinical isolate) and 15852 (clinical isolate). MICs were determined by microdilution susceptibility testing in 96-well plates (Nunc) using inocula of 10⁵ bacteria per ml. One hundred (100) μl of a bacterial suspension were added to 100 μl of culture medium (control) or to 100 μl of culture medium containing various antibiotic and C₁₂₍₅₋ₑₙₑ₎KKNC₁₂KNH₂ (SEQ ID NO: 2) concentrations in 2-fold serial dilutions. Inhibition of proliferation was determined by optical density measurements at 620 nm after an incubation period of 24 hours at 37° C.

TABLE 8

| | | Antibiotic MIC (μg/ml) in the presence | | | |
|---|---|---|---|---|---|
| | | of C₁₂₍₅₋ₑₙₑ₎KKNC₁₂KNH₂ (SEQ ID NO: 2) | | | |
| | | | +½ MIC | +⅓ MIC | +¼ MIC |
| Bacteria | Antibiotic | None | 3.12 μM | 2.1 μM | 1.6 μM |
| S. aureus 43300 | Oxacillin¹ | 0.5-1 | 0.06 | 0.125 | 0.125 |
| S. aureus 15819 | | 4 | 1 | 2 | 2 |
| S. aureus 15877 | | 64-128 | 16 | 32 | 64-128 |
| S. aureus 15852 | | 128 | 128 | 128 | 128 |
| S. aureus 43300 | Piperacillin¹ | 16 | 1 | 4 | 4 |
| S. aureus 15819 | | 16 | 4 | 8 | 8-16 |
| S. aureus 15877 | | 16-32 | 8-16 | 16-32 | 16-32 |
| S. aureus 15852 | | 128 | 128 | 128 | 128 |
| S. aureus 43300 | Penicillin G¹ | 4 | 0.03 | 0.25 | 1 |
| S. aureus 15819 | | 16 | 4 | 8 | 8-12 |
| S. aureus 15877 | | 8 | 8 | 8 | 8 |
| S. aureus 15852 | | 32 | 32 | 32 | 32 |
| S. aureus 43300 | Gentamicin² | 128 | 128 | 128 | 128 |
| S. aureus 15819 | | 1 | 1 | 1 | 1 |
| S. aureus 15877 | | 256 | 256 | 256 | 256 |
| S. aureus 15852 | | 512 | 512 | 512 | 512 |
| S. aureus 43300 | Tetracycline² | 0.25 | 0.06 | 0.125 | 0.125 |
| S. aureus 15819 | | 0.25 | 0.125 | 0.125 | 0.25 |
| S. aureus 15877 | | 1 | 0.5-1 | 1 | 1 |
| S. aureus 15852 | | 64-128 | 64-128 | 64-128 | 64-128 |
| S. aureus 43300 | Erythromycin² | 512 | 256 | 512 | 512 |
| S. aureus 15819 | | 0.25 | 0.25 | 0.25 | 0.25 |
| S. aureus 15877 | | >512 | >512 | >512 | >512 |
| S. aureus 15852 | | >512 | >512 | >512 | >512 |
| S. aureus 43300 | Ciprofloxacin³ | 0.5 | 0.03 | 0.125 | 0.25 |
| S. aureus 15819 | | 0.125-0.25 | 0.125 | 0.125-0.25 | 0.125-0.25 |
| S. aureus 15877 | | 4 | 4 | 4 | 4 |
| S. aureus 15852 | | 16 | 16 | 16 | 16 |

¹a classical antibiotic agent targeting cell wall biosynthesis;
²a classical antibiotic agent targeting protein synthesis;
³a classical antibiotic agent targeting DNA replication.

Table 8 reflects the polymer dose dependant re-sensitization of various bacterial strains towards various antibiotics, such as tetracycline and ciprofloxacin (known to act by distinct mechanisms: inhibition of ribosomal synthesis of proteins and DNA replication, respectively) as observed with some of the bacteria tested. As can be seen in Table 8, the data provide further support to results shown in Table 7 by demonstrating that the polymer can potentiate antibiotics effects over both resistant (Table 7) and sensitive strains (Table 8).

Table 9 below presents the MIC values of antibiotics in presence or absence of C₁₄₍₉₋ₑₙₑ₎KKNC₁₂KNH₂ (SEQ ID NO: 5) at sub-MIC levels of the polymer (MIC=6.2 μM), as measured for the S. aureus strains 17314 (clinical isolate) and 43300 (ATCC) and the E. coli strains 14384 (clinical isolate) and 16327 (clinical isolate). MIC values were determined by microdilution susceptibility testing in 96-well plates (Nunc) using inocula of 10⁵ bacteria per ml. 100 μl of a bacterial suspension were added to 100 μl of culture medium (control) or to 100 μl of culture medium containing various antibiotic and C₁₄₍₉₋ₑₙₑ₎KKNC₁₂KNH₂ (SEQ ID NO: 5) concentrations in 2-fold serial dilutions. Inhibition of proliferation was determined by optical density measurements at 620 nm after an incubation period of 24 hours at 37° C.

TABLE 9

| | | Antibiotic MIC (μg/ml) | | | |
|---|---|---|---|---|---|
| | | in the presence | | | |
| | | of C₁₄₍₉₋ₑₙₑ₎KKNC₁₂KNH₂ (SEQ ID NO: 5) | | | |
| | | | +½ MIC | +⅓ MIC | +¼ MIC |
| Bacteria | Antibiotic | None | 3.12 μM | 2.1 μM | 1.6 μM |
| E. coli 14384 | Oxacillin¹ | >512 | 256 | >512 | >512 |
| E. coli 16327 | | >512 | 512 | >512 | >512 |
| S. aureus 17314 | | 0.25 | 0.125-0.25 | 0.125-0.25 | 0.125-0.25 |
| S. aureus 43300 | | 0.5 | 0.25 | 0.25 | 0.25-0.5 |
| E. coli 14384 | | 256-512 | 256 | 256 | 256-512 |
| E. coli 16327 | Piperacillin¹ | 256 | 128 | 128 | 256 |
| S. aureus 17314 | | 32 | 32 | 32 | 32 |
| S. aureus 43300 | | 16 | 16 | 16 | 16 |
| E. coli 14384 | Penicillin G¹ | 512 | 512 | 512 | 512 |
| E. coli 16327 | | >512 | >512 | >512 | >512 |
| S. aureus 17314 | | 8 | 6 | 8 | 8 |
| S. aureus 43300 | | 4 | 2 | 4 | 4 |
| E. coli 14384 | Gentamicin² | 4 | 2 | 4 | 4 |
| E. coli 16327 | | 4 | 2 | 4 | 4 |
| S. aureus 17314 | | 512 | 512 | 512 | 512 |
| S. aureus 43300 | | 64-128 | 64-128 | 64-128 | 64-128 |
| E. coli 14384 | Tetracycline² | 128-256 | 32 | 128 | 128 |
| E. coli 16327 | | >512 | 32 | 256 | 512 |
| S. aureus 17314 | | 0.25 | 0.25 | 0.25 | 0.25 |
| S. aureus 43300 | | 0.25 | 0.125 | 0.125-0.25 | 0.125-0.25 |
| E. coli 14384 | Erythromycin² | 128 | 32 | 64 | 64-128 |
| E. coli 16327 | | 256 | 64 | 256 | 256 |
| S. aureus 17314 | | 1 | 1 | 1 | 1 |
| S. aureus 43300 | | >512 | >512 | >512 | >512 |
| E. coli 14384 | Ciprofloxacin³ | <0.03 | not | not | not |
| | | | determined | determined | determined |
| E. coli 16327 | | >512 | 32-64 | 64-128 | 128-256 |
| S. aureus 17314 | | 64 | 64 | 64 | 64 |
| S. aureus 43300 | | 0.5 | 0.25 | 0.25-0.5 | 0.25-0.5 |

¹a classical antibiotic agent targeting cell wall biosynthesis;
²a classical antibiotic agent targeting protein synthesis;
³a classical antibiotic agent targeting DNA replication.

Table 9 reflects the polymer's dose dependant re-sensitization of various bacterial strains towards various antibiotics, such as tetracycline and ciprofloxacin (known to act by distinct mechanisms: inhibition of ribosomal synthesis of proteins and DNA replication, respectively) as observed mainly against E. coli strains. Thus, along with results listed in Table 8 and additional results presented hereinbelow, these data provide a structure-activity relationships study demonstrating the importance of the N-terminal acyl on the said activities.

Table 10 below presents the MIC values of antibiotics in presence or absence of C₁₆₍₉₋ₑₙₑ₎KNC₁₂KNH₂ (SEQ ID NO: 6) at sub-MIC levels of the polymer (MIC=6.2 μM), as measured for S. aureus 17314 and 43300, E. coli 16327 and 16329, and P. aeruginosa 8732. MIC values were determined as described for Tables 8 and 9 hereinabove.

Table 10 reflects the polymer's dose dependant re-sensitization of various bacterial strains towards various antibiotics, particularly β-lactam (cell-wall targeting) antibiotics as observed mainly against S. aureus strains.

TABLE 10

| | | Antibiotic MIC (μg/ml) | | | |
|---|---|---|---|---|---|
| | | in the presence | | | |
| | | of C₁₆₍₉₋ₑₙₑ₎KKNC₁₂KNH₂ (SEQ ID NO: 6) | | | |
| | | | +½ MIC | +⅓ MIC | +¼ MIC |
| Bacteria | Antibiotic | None | 3.12 μM | 2.1 μM | 1.6 μM |
| S. aureus 17314 | Oxacillin¹ | 0.25-0.5 | 0.06 | 0.25 | 0.25 |
| S. aureus 43300 | | 0.5-1 | 0.03-0.06 | 0.25-0.125 | 0.25-0.5 |
| E. coli 16327 | | >512 | >512 | >512 | >512 |
| E. coli 16329 | | >512 | >512 | >512 | >512 |
| P. aeruginosa 8732 | | >512 | >512 | >512 | >512 |
| S. aureus 17314 | Piperacillin¹ | 8 | 1 | 2 | 4 |
| S. aureus 43300 | | 16 | 2 | 4 | 4 |
| E. coli 16327 | | 512 | 256 | 256 | 256 |
| E. coli 16329 | | 256 | 128-256 | 128-256 | 128-256 |
| P. aeruginosa 8732 | | 256 | 256 | 256 | 256 |
| S. aureus 17314 | Penicillin G¹ | 8-16 | 0.5 | 1 | 2 |
| S. aureus 43300 | | 4 | 0.25 | 1-2 | 2 |
| E. coli 16327 | | >512 | >512 | >512 | >512 |
| E. coli 16329 | | 512 | 256 | 256 | 256 |
| P. aeruginosa 8732 | | 512 | 512 | 512 | 512 |
| S. aureus 17314 | Gentamicin² | 256 | 64 | 128 | 256 |
| S. aureus 43300 | | 64 | 32-64 | 64 | 64 |
| E. coli 16327 | | 4 | 4 | 4 | 4 |
| E. coli 16329 | | 512 | 512 | 512 | 512 |
| P. aeruginosa 8732 | | 4 | 4 | 4 | 4 |
| S. aureus 17314 | Tetracycline² | 0.25 | 0.06-0.125 | 0.125-0.25 | 0.125-0.25 |
| S. aureus 43300 | | 0.25 | 0.125 | 0.125 | 0.25 |
| E. coli 16327 | | >512 | >512 | >512 | >512 |
| E. coli 16329 | | 256 | 128-256 | 128-256 | 128-256 |
| P. aeruginosa 8732 | | 256 | 256 | 256 | 256 |
| S. aureus 17314 | Erythromycin² | 1 | 0.5 | 1 | 1 |
| S. aureus 43300 | | 512 | 512 | 512 | 512 |
| E. coli 16327 | | 256 | 256 | 256 | 256 |
| E. coli 16329 | | 256 | 128 | 256 | 256 |
| P. aeruginosa 8732 | | 128 | 128 | 128 | 128 |
| S. aureus 17314 | Ciprofloxacin³ | 64 | 16 | 32 | 32 |
| S. aureus 43300 | | 0.25 | 0.125 | 0.25 | 0.25 |
| E. coli 16327 | | >512 | >512 | >512 | >512 |
| E. coli 16329 | | 64 | 64 | 64 | 64 |
| P. aeruginosa 8732 | | <0.03 | not | not | not |
| | | | determined | determined | determined |

¹a classical antibiotic agent targeting cell wall biosynthesis;
²a classical antibiotic agent targeting protein synthesis;
³a classical antibiotic agent targeting DNA replication.

Table 11 below presents the MIC values of antibiotics in presence or absence of C₁₂KKNC₁₂KNH₂ (SEQ ID NO: 7) at sub-MIC levels of the polymer, as measured for the S. aureus strains 43300, 15819, 17314 and 15852. MIC values were determined as described for Tables 8, 9 and 10 hereinabove.

TABLE 11

| | | Antibiotic MIC (μg/ml) | | | |
|---|---|---|---|---|---|
| | | In presence of C₁₂KKNC₁₂KNH₂ (SEQ ID NO: 7) | | | |
| | | | +½ MIC | +⅓ MIC | +¼ MIC |
| Bacteria | Antibiotic | None | 3.12 μM | 2.1 μM | 1.6 μM |
| S. aureus 43300 | Oxacillin | 0.5 | 0.25 | 0.25.0.5 | 0.5 |
| S. aureus 15819 | | 8 | 2 | 8 | 8 |
| S. aureus 17314 | | 0.25 | 0.25 | 0.25 | 0.25 |
| S. aureus 15852 | | 256 | 256 | 256 | 256 |
| S. aureus 43300 | Piperacillin | 32 | 8 | 16 | 32 |
| S. aureus 15819 | | 32 | 4 | 8-16 | 16 |
| S. aureus 17314 | | 128 | 64 | 128 | 128 |
| S. aureus 15852 | | 256 | 256 | 256 | 256 |
| S. aureus 43300 | Penicillin G | 8 | 1 | 2-4 | 4 |
| S. aureus 15819 | | 16 | 2 | 4 | 8 |
| S. aureus 17314 | | 128 | 64 | 64-128 | 64-128 |
| S. aureus 15852 | | 64 | 64 | 64 | 64 |
| S. aureus 43300 | Gentamicin | 256 | 256 | 256 | 256 |
| S. aureus 15819 | | 1 | 1 | 1 | 1 |
| S. aureus 17314 | | 512 | 512 | 512 | 512 |
| S. aureus 15852 | | >512 | >512 | >512 | >512 |
| S. aureus 43300 | Tetracycline | 0.5 | 0.5 | 0.5 | 0.5 |
| S. aureus 15819 | | 0.5 | 0.5 | 0.5 | 0.5 |
| S. aureus 17314 | | 0.25-0.5 | 0.25-0.5 | 0.25-0.5 | 0.25-0.5 |
| S. aureus 15852 | | 64-128 | 64-128 | 64-128 | 64-128 |
| S. aureus 43300 | Erythromycin | >512 | >512 | >512 | >512 |
| S. aureus 15819 | | 0.5 | 0.5 | 0.5 | 0.5 |
| S. aureus 17314 | | 1 | 1 | 1 | 1 |
| S. aureus 15852 | | >512 | >512 | >512 | >512 |
| S. aureus 43300 | Ciprofloxacin | 0.5 | 0.25 | 0.5 | 0.5 |
| S. aureus 15819 | | 0.125-0.25 | 0.125-0.25 | 0.125-0.25 | 0.125-0.25 |
| S. aureus 17314 | | 128 | 64 | 64 | 128 |
| S. aureus 15852 | | 32 | 32 | 32 | 32 |

Table 11 reflects the polymer's dose dependant re-sensitization of several S. aureus strains towards β-lactam (cell-wall targeting) antibiotics.

Table 12 below presents the MIC values of antibiotics in presence or absence of C₁₂K(KNC₁₂K)₂NH₂ (SEQ ID NO: 8) at sub-MIC levels of the polymer, as measured for P. mirabilis 1285, E. coli 16327, and S. aureus 17314 and 43300. MIC values were determined as described for Tables 8, 9, 10 and 11 hereinabove.

TABLE 12

| | | Antibiotic MIC (μg/ml) | | | |
|---|---|---|---|---|---|
| | | In presence of C₁₂K(KNC₁₂K)₂NH₂ | | | |
| | | (SEQ ID NO: 8) | | | |
| | | | +½ MIC | +⅓ MIC | +¼ MIC |
| Bacteria | Antibiotic | None | 3.12 μM | 2.1 μM | 1.6 μM |
| P. mirabilis 1285 | Oxacillin | >512 | 512 | >512 | >512 |
| E. coli 16327 | | >512 | 512 | >512 | >512 |
| S. aureus 17314 | | 0.25 | 0.125-0.25 | 0.125-0.25 | 0.125-0.25 |
| S. aureus 43300 | | 0.5 | 0.25 | 0.25 | 0.25-0.5 |
| P. mirabilis 1285 | Piperacillin | 512 | 512 | 512 | 512 |
| E. coli 16327 | | 256 | 64 | 256 | 256 |
| S. aureus 17314 | | 128 | 64 | 128 | 128 |
| S. aureus 43300 | | 32 | 8-16 | 32 | 32 |
| P. mirabilis 1285 | Penicillin G | 512 | 512 | 512 | 512 |
| E. coli 16327 | | >512 | >512 | >512 | >512 |
| S. aureus 17314 | | 128 | 64 | 128 | 128 |
| S. aureus 43300 | | 8 | 4 | 8 | 8 |
| P. mirabilis 1285 | Gentamicin | 32 | 32 | 32 | 32 |
| E. coli 16327 | | 8 | 4 | 8 | 8 |
| S. aureus 17314 | | 512 | 512 | 512 | 512 |
| S. aureus 43300 | | 256 | 256 | 256 | 256 |
| P. mirabilis 1285 | Tetracycline | 256 | 32 | 64 | 64 |
| E. coli 16327 | | 512 | 128-256 | 512 | 512 |
| S. aureus 17314 | | 0.25-0.5 | 0.25 | 0.5 | 0.25 |
| S. aureus 43300 | | 0.5 | 0.5 | 0.5 | 0.5 |
| P. mirabilis 1285 | Erythromycin | >512 | 128 | 256 | 512 |
| E. coli 16327 | | 256 | 128-256 | 256 | 256 |
| S. aureus 17314 | | 1 | 0.5-1 | 0.5-1 | 1 |
| S. aureus 43300 | | >512 | >512 | >512 | >512 |
| P. mirabilis 1285 | Ciprofloxacin | 32 | 8 | 16 | 32 |
| E. coli 16327 | | >512 | 128 | >512 | >512 |
| S. aureus 17314 | | 128 | 64 | 64 | 128 |
| S. aureus 43300 | | 0.5 | 0.13-0.25 | 0.25-0.5 | 0.25-0.5 |

Table 13 below presents the MIC values of antibiotics in presence or absence of C₁₂K(KNC₁₂K)₃NH₂ (SEQ ID NO: 9) at sub-MIC levels of the polymer, as measured for S. maltophila 748 and S. aureus 15819. MIC values were determined as described for Tables 8, 9 and 10 hereinabove.

As can be seen in Table 13, the data indicate that C₁₂K(KNC₁₂K)₃NH₂ (SEQ ID NO: 9) is capable of re-sensitizing two bacterial species ad strains towards various antibiotics known to act by distinct mechanisms, against both tested strains.

TABLE 13

| | | Antibiotic MIC (μg/ml) | | | |
|---|---|---|---|---|---|
| | | In presence of C₁₂K(KNC₁₂K)₃NH₂ | | | |
| | | (SEQ ID NO: 9) | | | |
| | | | +½ | | |
| | | | MIC | +⅓ | +¼ |
| | | | 3.12 | MIC | MIC |
| Bacteria | Antibiotic | None | μM | 2.1 μM | 1.6 μM |
| S. maltophila | Oxacillin | 512 | 256 | 512 | 512 |
| 748 | | | | | |
| S. aureus 15819 | | 8 | 0.5-1 | 2 | 4 |
| S. maltophila | Piperacillin | 32 | 32 | 32 | 32 |
| 748 | | | | | |
| S. aureus 15819 | | 32 | 4 | 4 | 8-16 |
| S. maltophila | Penicillin G | 512 | 128 | 256-512 | 512 |
| 748 | | | | | |
| S. aureus 15819 | | 16 | 0.5 | 2 | 4 |
| S. maltophila | Gentamicin | 16 | 8 | 16 | 16 |
| 748 | | | | | |
| S. aureus 15819 | | 1 | 0.5 | 1 | 1 |
| S. maltophila | Tetracycline | 128 | 32 | 64 | 64 |
| 748 | | | | | |
| S. aureus 15819 | | 0.5 | 0.125 | 0.25 | 0.5 |
| S. maltophila | Erythromycin | 512 | 256 | 512 | 512 |
| 748 | | | | | |
| S. aureus 15819 | | 0.5 | 0.125 | 0.25 | 0.5 |
| S. maltophila | Ciprofloxacin | 8 | 2 | 4-8 | 8 |
| 748 | | | | | |
| S. aureus 15819 | | 0.25 | 0.06 | 0.125 | 0.125 |

Re-Sensitizing Effect Against Gram Negative Bacteria:

The re-sensitizing effect of exemplary antimicrobial re-sensitizing polymers against gram negative bacteria, such as E. coli, that are normally insensitive to oxacillin (at least up to 512 μg/ml) but sensitive to the exemplary polymers, such as C₁₂K(NC₈K)₇NH₂ (SEQ ID NO: 4) (also referred to as C₁₂K-7α₈) and C₁₂K(NC₈K)₅NH₂ (SEQ ID NO: 3) (also referred to as C₁₂K-5α₈) having MIC values of 3.1 μM and 6.2 μM, respectively), was tested in similar means as presented hereinabove.

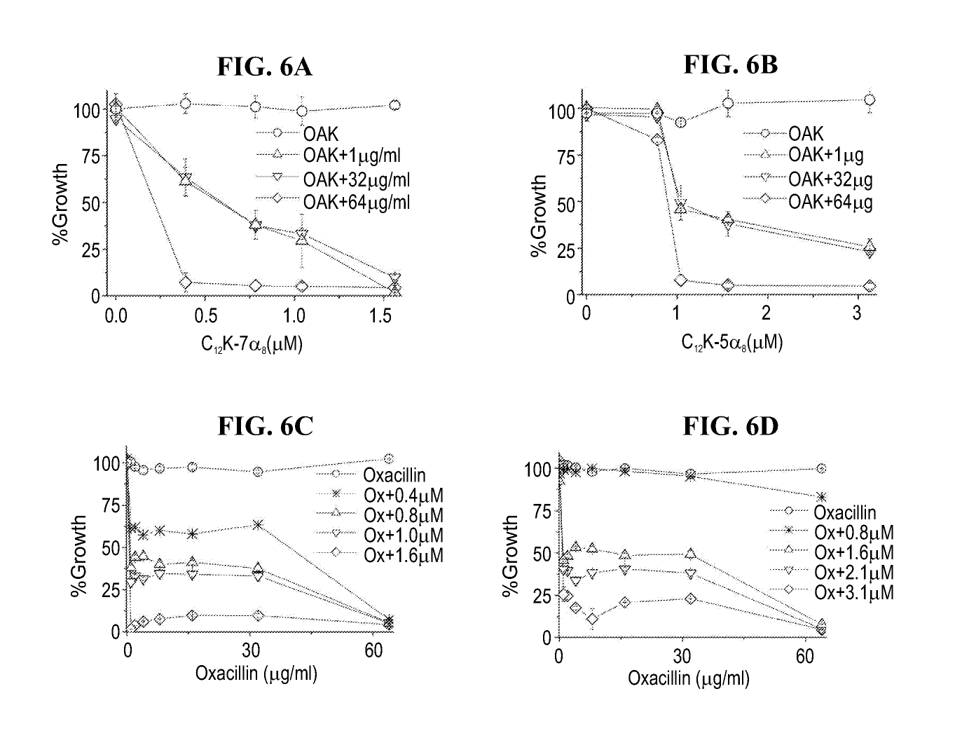

FIGS. 6A-6D present the results of the experiments demonstrating the antimicrobial re-sensitizing effect of C₁₂K(NC₈K)₇NH₂ (SEQ ID NO: 4) and C₁₂K(NC₈K)₅NH₂ (SEQ ID NO: 3), two exemplary polymers according to some embodiments presented herein, in combination with oxacillin, as assessed against E. coli CI14213 strain after 24 hours incubation, wherein FIGS. 6A-B show that the polymers' activity was improved in presence of oxacillin, and wherein FIGS. 6C-6D show that while oxacillin alone was inactive against E. coli, the addition of the polymers at concentrations well below their MIC value (up to ⅛ MIC) has endowed potency to oxacillin.

As can be seen in FIGS. 6A-6D, both polymers are capable of potentiating oxacillin and further show that oxacillin is also capable of potentiating the polymer's effect, albeit to a lesser extent and more so for C₁₂K(NC₈K)₇NH₂ than C₁₂K(NC₈K)₅NH₂. As can further be seen in FIGS. 6A-6D, the polymer presented herein posses the ability of to broaden the spectrum of activity of the tested antibiotics, as oxacillin is normally not indicated for treatment of Gram negative bacteria.

### Combinations of Non-Polymer Antimicrobial Agents and Various Antimicrobial Re-Sensitizing Polymers Against Clinical Isolates of Escherichia Coli:

The results presented below demonstrate the re-sensitizing activity of some exemplary antimicrobial re-sensitizing polymers, C₁₂K(NC₈K)₅NH₂ (SEQ ID NO: 3) (Tables 14 and 16 hereinbelow) and C₁₂K(NC₈K)₇NH₂ (SEQ ID NO: 4) (Tables 15 and 17 hereinbelow), according to embodiments of the present invention, when acting together with a series of classical antibiotic agents, such as oxacillin, piperacillin, penicillin G, ciprofloxacin, erythromycin, tetracycline, gentamicin and methicillin, against clinical isolates of Escherichia coli strains.

Tables 14 and 15 below present the results obtained for combinations of seven different classical antibiotic agents and the polymers C₁₂K(NC₈K)₇NH₂ (SEQ ID NO: 4) and C₁₂K(NC₈K)₅NH₂ (SEQ ID NO: 3) against clinical isolates of Escherichia coli, wherein the MICs determined for all strains were similar, namely 3.1 μM (corresponding to 7.0 μg/ml for C₁₂K(NC₈K)₇NH₂ (SEQ ID NO: 4) and 5.2 μg/ml for C₁₂K(NC₈K)₅NH₂ (SEQ ID NO: 3)).

TABLE 14

| | | | Antibiotic MIC (μg/ml) in presence of | | | |
|---|---|---|---|---|---|---|
| | | E. coli | C₁₂K(NC₈K)₅NH₂ (SEQ ID NO: 3) | | | |
| Antibiotic | | Strain | None | +½ MIC | +⅓ MIC | +¼ MIC |
| Protein | Erythromycin | 14182 | 64-128 | 8-16 | 32-64 | 64 |
| synthesis | | 14384 | 128-256 | 4-8 | 32 | 32-64 |
| | | U-16329 | 128-256 | 32 | 64 | 128 |
| | Tetracycline | 14182 | 32-64 | 8-16 | 32 | 32 |
| | | 14384 | 128-256 | 32-64 | 128 | 128 |
| | | U-16329 | 128-256 | 64 | 128 | 128 |
| | Gentamycin | 14182 | 128 | 128 | Not | Not |
| | | | | | Determined | Determined |
| | | 14384 | 4 | 1-2 | Not | Not |
| | | | | | Determined | Determined |
| | | U-16329 | >512 | >512 | >512 | >512 |
| Cell-wall | Piperacillin | 14182 | >512 | 512 | >512 | >512 |
| Biosynthesis | | 14384 | 256-512 | 256-512 | Not | Not |
| | | | | | Determined | Determined |
| | | U-16329 | 256 | 128 | 128-256 | 256 |
| | Penicillin G | 14182 | >512 | >512 | Not | Not |
| | | | | | Determined | Determined |
| | | 14384 | >512 | >512 | Not | Not |
| | | | | | Determined | Determined |
| | | U-16329 | 256 | 256 | 256 | 256 |
| | Oxacillin | 14182 | >512 | >512 | Not | Not |
| | | | | | Determined | Determined |
| | | 14384 | >512 | 256-512 | Not | Not |
| | | | | | Determined | Determined |
| | | U-16329 | >512 | >512 | >512 | >512 |
| DNA | Ciprofloxacin | 14182 | <0.03 | Not | Not | Not |
| replication | | | | Determined | Determined | Determined |
| | | 14384 | <0.03 | Not | Not | Not |
| | | | | Determined | Determined | Determined |
| | | U-16329 | 64 | 32 | 64 | 64 |

TABLE 15

| | | | Antibiotic MIC (μg/ml) in presence of | | | |
|---|---|---|---|---|---|---|
| | | E. coli | C₁₂K(NC₈K)₇NH₂ (SEQ ID NO: 4) | | | |
| Antibiotic | | Strain | None | +½ MIC | +⅓ MIC | +¼ MIC |
| Protein | Erythromycin | 14182 | 64-128 | 8 | 16 | 32 |
| synthesis | | 14384 | 128-256 | 8-16 | 16-32 | 64-128 |
| | | U-16329 | 128-256 | 16 | 128 | 128 |
| | | U-16327 | 128-256 | 32 | 64-128 | 128 |
| | Tetracycline | 14182 | 32-64 | 8-16 | 32 | 32 |
| | | 14384 | 128-256 | 64 | 128 | 128-256 |
| | | U-16329 | 128-256 | 32-64 | 128 | 128 |
| | | U-16327 | >512 | 32 | 256 | 512 |
| | Gentamycin | 14182 | 128 | 64-128 | 128 | 128 |
| | | 14384 | 4 | 2 | 2-4 | 4 |
| | | U-16329 | >512 | >512 | >512 | >512 |
| | | U-16327 | 4 | 1-2 | 2 | 4 |
| Cell-wall | Piperacillin | 14182 | >512 | 64 | >512 | >512 |
| Biosynthesis | | 14384 | 256-512 | 64-128 | 256-512 | 256-512 |
| | | U-16329 | 256 | 64-128 | 128-256 | 128-256 |
| | | U-16327 | 256 | 32-64 | 128-256 | 256 |
| | Penicillin G | 14182 | >512 | >512 | >512 | >512 |
| | | 14384 | >512 | >512 | >512 | >512 |
| | | U-16329 | 256 | 128-256 | 128-256 | 256 |
| | | U-16327 | >512 | 512 | >512 | >512 |
| | Oxacillin | 14182 | >512 | >512 | >512 | >512 |
| | | 14384 | >512 | >512 | >512 | >512 |
| | | U-16329 | >512 | 512 | >512 | >512 |
| | | U-16327 | >512 | 512 | >512 | >512 |
| DNA | Ciprofloxacin | 14182 | <0.03 | Not | Not | Not |
| repliction | | | | Determined | Determined | Determined |
| | | 14384 | <0.03 | Not | Not | Not |
| | | | | Determined | Determined | Determined |
| | | U-16329 | 64 | 64 | 64 | 64-128 |
| | | U-16327 | 128-256 | 16 | 32-64 | 64-128 |

As can be seen in Tables 14 and 15, when assessed at various sub-MIC values both polymers (C₁₂K(NC₈K)₇NH₂ (SEQ ID NO: 4) and C₁₂K(NC₈K)₅NH₂ (SEQ ID NO: 3), respectively) enhanced in a dose-dependent manner, the effect of several antibiotics (by up to 32 folds) including erythromycin and tetracycline, whose resistance mechanism often involves efflux pumps.

Tables 16 and 17 below present the results obtained for combinations of seven different classical antibiotic agents and the polymers C₁₂K(NC₈K)₇NH₂ (SEQ ID NO: 4) and C₁₂K(NC₈K)₅NH₂ (SEQ ID NO: 3) (MIC=3.125 μg/ml) against an isogenic pair of E. coli K-12 strains (a resistant wild type and an efflux knockout mutant), wherein the polymers enhanced antibiotics potency against the multi-resistant strain only, while in the strain with decreased levels of efflux pumps, the MICs were not altered significantly.

TABLE 16

| | | | Antibiotic MIC (μg/ml) in presence of | | | |
|---|---|---|---|---|---|---|
| | | | C₁₂K(NC₈K)₇NH₂ (SEQ ID NO: 4) | | | |
| Antibiotic | | Strain | None | +½ MIC | +⅓ MIC | +¼ MIC |
| Protein | Erythromycin | E. coli | 128 | 8-16 | 64 | 128 |
| synthesis | | AG100 | | | | |
| | | (w.t.) | | | | |
| | | E. coli | 4-8 | 2 | 2-4 | 4-8 |
| | | AG100A | | | | |
| | | (efflux−) | | | | |
| | Tetracycline | E. coli | 2 | 0.25-0.5 | 0.5 | 1-2 |
| | | AG100 | | | | |
| | | (w.t.) | | | | |
| | | E. coli | 0.5 | 0.25-0.5 | 0.5 | 0.5 |
| | | AG100A | | | | |
| | | (efflux−) | | | | |
| | Gentamycin | E. coli | 4-8 | 2 | 4 | 4 |
| | | AG100 | | | | |
| | | (w.t.) | | | | |
| | | E. coli | 4-8 | 2-4 | 4 | 4 |
| | | AG100A | | | | |
| | | (efflux−) | | | | |
| Cell-wall | Piperacillin | E. coli | 2 | 0.5 | 1 | 2 |
| Biosynthesis | | AG100 | | | | |
| | | (w.t.) | | | | |
| | | E. coli | <0.13 | <0.13 | <0.13 | <0.13 |
| | | AG100A | | | | |
| | | (efflux−) | | | | |
| | Penicillin G | E. coli | 32 | 8 | 8-16 | 16-32 |
| | | AG100 | | | | |
| | | (w.t.) | | | | |
| | | E. coli | 8 | 4 | 8 | 8 |
| | | AG100A | | | | |
| | | (efflux−) | | | | |
| | Oxacillin | E. coli | 512 | 32-64 | 64-128 | 128 |
| | | AG100 | | | | |
| | | (w.t.) | | | | |
| | | E. coli | 0.5-1 | 0.25 | 0.25 | 0.5-1 |
| | | AG100A | | | | |
| | | (efflux−) | | | | |
| DNA | Ciprofloxacin | E. coli | <0.06 | — | — | — |
| replication | | AG100 | | | | |
| | | (w.t.) | | | | |
| | | E. coli | <0.06 | — | — | — |
| | | AG100A | | | | |
| | | (efflux−) | | | | |

TABLE 17

| | | | Antibiotic MIC (μg/ml) in presence of the OAK | | | |
|---|---|---|---|---|---|---|
| Antibiotic | | Strain | None | +½ MIC | +⅓ MIC | +¼ MIC |
| Protein | Erythromycin | E. coli | 128 | 16 | 64 | 64-128 |
| synthesis | | AG100 | | | | |
| | | (w.t.) | | | | |
| | | E. coli | 4-8 | 1-2 | 4 | 4-8 |
| | | AG100A | | | | |
| | | (efflux−) | | | | |
| | Tetracycline | E. coli | 2 | 0.5 | 1 | 2 |
| | | AG100 | | | | |
| | | (w.t.) | | | | |
| | | E. coli | 0.5 | 0.5 | 0.5 | 0.5 |
| | | AG100A | | | | |
| | | (efflux−) | | | | |
| | Gentamycin | E. coli | 4-8 | 4 | 4 | 4 |
| | | AG100 | | | | |
| | | (w.t.) | | | | |
| | | E. coli | 4-8 | 4 | 4 | 4 |
| | | AG100A | | | | |
| | | (efflux−) | | | | |
| Cell-wall | Piperacillin | E. coli | 2 | 0.5 | 1 | 1-2 |
| Biosynthesis | | AG100 | | | | |
| | | (w.t.) | | | | |
| | | E. coli | <0.13 | <0.13 | <0.13 | <0.13 |
| | | AG100A | | | | |
| | | (efflux−) | | | | |
| | Penicillin G | E. coli | 32 | 8 | 16 | 16 |
| | | AG100 | | | | |
| | | (w.t.) | | | | |
| | | E. coli | 8 | 4 | 4-8 | 4-8 |
| | | AG100A | | | | |
| | | (efflux−) | | | | |
| | Oxacillin | E. coli | 512 | 32-64 | 128 | 256 |
| | | AG100 | | | | |
| | | (w.t.) | | | | |
| | | E. coli | 0.5-1 | 0.25 | 0.5 | 0.5 |
| | | AG100A | | | | |
| | | (efflux−) | | | | |
| DNA | Ciprofloxacin | E. coli | <0.06 | — | — | — |
| replication | | AG100 | | | | |
| | | (w.t.) | | | | |
| | | E. coli | <0.06 | — | — | — |
| | | AG100A | | | | |
| | | (efflux−) | | | | |

As can be seen in Tables 16 and 17, sub-inhibitory polymer concentrations may help re-sensitize antibiotic-resistant bacteria including those whose main resistance mechanism involves increased levels of active efflux pumps.

Tables 18 and 19 below present the results obtained for combinations of erythromycin and the polymers C₁₂K(NC₈K)₇NH₂ (SEQ ID NO: 4) and C₁₂K(NC₈K)₅NH₂ (SEQ ID NO: 3) respectively, against erythromycin resistant E. coli K-12 strains (resistant due to a mutation in the ribosomal protein), wherein E. coli N281 has a mutation in the ribosomal proteins L22 (eryB) which is a deletion of three amino acid residues, Met82, Lys83 and Arg84, and E. coli N282 has a mutation in the ribosomal proteins L4 (eryA) which is a single amino acid substitution, Lys63Glu. The polymer C₁₂K(NC₈K)₇NH₂ (SEQ ID NO: 4) exhibited a MIC on N281 of 3.1 μM and a MIC on N282 of 1.6 μM, and the polymer C₁₂K(NC₈K)₇NH₂ (SEQ ID NO: 4) exhibited a MIC on N281 of 3.1 μM and MIC on N282 of 3.1 μM.

TABLE 18

| | | Antibiotic MIC (μg/ml) in presence | | | |
|---|---|---|---|---|---|
| | | of C₁₂K(NC₈K)₇NH₂ (SEQ ID NO: 4) | | | |
| | | erythromycin + | erythromycin + | erythromycin + | erythromycin + |
| Strain | Genotype | no OAK | ½ MIC | ⅓ MIC | ¼ MIC |
| E. coli | Wild type | 64-128 | 4-8 | 16-32 | 32 |
| AB301 | | | | | |
| E. coli | L22 mutation | >512 | 128 | 512 | >512-512 |
| N281 | | | | | |
| E. coli | L4 mutation | 512 | 256-512 | 512 | 512 |
| N282 | | | | | |

TABLE 19

| | | Antibiotic MIC (μg/ml) in presence | | | |
|---|---|---|---|---|---|
| | | of C₁₂K(NC₈K)₅NH₂ (SEQ ID NO: 3) | | | |
| | | erythromycin + | erythromycin + | erythromycin + | erythromycin + |
| Strain | Genotype | no OAK | ½ MIC | ⅓ MIC | ¼ MIC |
| E. coli | Wild type | 64-128 | 8 | 16-32 | 32 |
| AB301 | | | | | |
| E. coli | L22 mutation | >512 | 16-32 | 128 | 512 |
| N281 | | | | | |
| E. coli | L4 mutation | 512 | 8-16 | 256 | 512 |
| N282 | | | | | |

As can be seen in Tables 18 and 19, the polymers enhanced antibiotics potency against the multi-resistant strain only, while in the strain with decreased levels of efflux pumps, the MICs were not altered significantly. The reason for this differential behavior can be attributed to the polymers' distinct mechanism of action involving either inhibition of DNA functions and plasma membrane disruption, respectively (Rotem et al., 2008 FASEB J. 22:2652-2661).

Tables 20 and 21 below present the results obtained for combinations of the classical antibiotics piperacillin, penicillin G, oxacillin and ampicillin and the polymers C₁₂K(NC₈K)₇NH₂ (SEQ ID NO: 4) and C₁₂K(NC₈K)₅NH₂ (SEQ ID NO: 3) respectively, against over expressing beta lactamase E. coli K-12 strains, wherein a plasmid encoding for beta lactamse production was inserted into a wild type strain (E. coli AG100) resulting with a resistant mutant (E. coli AG100/ks), and against two additional resistant strains, which were obtained from Coli Genetic Stock Center, namely E. coli D21 and E. coli G11a1 which produce about 10 fold the amount of beta lactamase found in corresponding wild-type strains. The polymers exhibited a MIC of 3.1 μM against all strains presented below.

TABLE 20

| | | Antibiotic MIC (μg/ml) in presence | | | |
|---|---|---|---|---|---|
| | | of C₁₂K(NC₈K)₇NH₂ (SEQ ID NO: 4) | | | |
| Antibiotic | Strain | None | +½ MIC | +⅓ MIC | +¼ MIC |
| Piperacillin | E. coli AG100 | 2 | 0.5 | 1 | 2 |
| | E. coli | >512 | >512-512 | >512 | >512 |
| | AG100/ks | | | | |
| Penicillin G | E. coli AG100 | 32 | 8 | 8-16 | 16-32 |
| | E. coli | >512 | >512 | >512 | >512 |
| | AG100/ks | | | | |
| | E. coli G11a1 | 512 | 256 | 256 | 512 |
| | E. coli D21 | 512 | 256 | 512 | 512 |
| Oxacillin | E. coli AG100 | 512 | 32-64 | 64-128 | 128 |
| | E. coli | >512 | >512 | >512 | >512 |
| | AG100/ks | | | | |
| Ampicillin | E. coli AG100 | 4 | — | — | — |
| | E. coli | >512 | >512 | >512 | >512 |
| | AG100/ks | | | | |
| | E. coli G11a1 | 64 | 32 | 64 | 64 |
| | E. coli D21 | 64 | 32 | 64 | 64 |

TABLE 21

| | | Antibiotic MIC (μg/ml) in presence | | | |
|---|---|---|---|---|---|
| | | of C₁₂K(NC₈K)₅NH₂ (SEQ ID NO: 3) | | | |
| Antibiotic | Strain | None | +½ MIC | +⅓ MIC | +¼ MIC |
| Piperacillin | E. coli AG100 | 2 | 0.5 | 1 | 1-2 |
| | E. coli | >512 | >512-512 | >512 | >512 |
| | AG100/ks | | | | |
| Penicillin G | E. coli AG100 | 32 | 8 | 16 | 16 |
| | E. coli | >512 | >512 | >512 | >512 |
| | AG100/ks | | | | |
| | E. coli G11a1 | 512 | 256-512 | 512 | 512 |
| | E. coli D21 | 512 | 256 | 256-512 | 512 |
| Oxacillin | E. coli AG100 | 512 | 32-64 | 128 | 256 |
| | E. coli | >512 | >512 | >512 | >512 |
| | AG100/ks | | | | |
| Ampicillin | E. coli AG100 | 4 | — | — | — |
| | E. coli | >512 | >512 | >512 | >512 |
| | AG100/ks | | | | |
| | E. coli G11a1 | 64 | 32 | 64 | 64 |
| | E. coli D21 | 64 | 32 | 64 | 64 |

As can be seen in Tables 20 and 21, in presence of E. coli K-12 strains whose main resistance mechanism involves the over expression of beta lactamase, the re-sensitization effect of antibiotics is negligible (did not yield significant MIC reductions) by neither of the two polymers.

Table 22 below presents the results obtained for combinations of the classical antibiotics cefazolin, cefoperazone, cefotaxime, ciproflaxacin, erythromycin, gentamicin sulfate, oxacillin, penicillin G, piperacillin, tetracycline and vancomycin, and the polymer C₁₂K(KNC₁₀K)₃NH₂ (SEQ ID NO: 10) (also referred to as C₁₂K-3β₁₀), against MRSA 15903 (MIC of polymer 6.25 μM) and E.coli U16327 (MIC of polymer 3.1 μM). MIC values were determined as described hereinabove.

TABLE 22

| | | MIC (μg/ml) | | | |
|---|---|---|---|---|---|
| | | | ½ + MIC | ⅓ + MIC | ¼ + MIC |
| | Antibiotic | Alone | OAK | OAK | OAK |
| MRSA | Oxacillin | 128-64 | 0.5 | 16-64 | 128-64 |
| 15903 | piperacillin | 256-128 | 32-8 | 256-128 | 256-128 |
| | Penicillin G | 64 | 4-16 | 64 | 64 |
| | Cefazolin | 16 | 2-4 | 8-16 | 16 |
| | Cefoperazone | 16 | 4 | 8-16 | 16 |
| | Cefotaxime | 32 | 4 | 16 | 32 |
| | Erythromycin | 1 | 0.13-0.25 | 0.5 | 1-0.5 |
| | Tetracycline | 1 | 0.25 | 1 | 1 |
| | Gentamicin | >256 | 256 | >256 | >256 |
| | Sulfate | | | | |
| | Vancomycin | 1 | 1 | 1 | 1 |
| | Ciproflaxacin | 256 | 64 | 128 | 128 |
| E. coli | Oxacillin | >512 | 512-256 | >512 | >512 |
| U16327 | Penicillin G | >512 | 512 | >512 | >512 |
| | Erythromycin | 256 | 4-8 | 8 | 256 |
| | Gentamicin | 4 | 2-4 | 4 | 4 |
| | Sulfate | | | | |
| | Tetracycline | 512 | 64 | 128-256 | 512 |
| | Ciproflaxacin | 256 | 128 | 128 | 128-256 |

As can be seen in Table 22, the data indicates that C₁₂K(KNC₁₀K)₃NH₂ (SEQ ID NO: 10) is able to potentiate various antibiotics known to act by distinct mechanisms, against both S. aureus and E. coli strains.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of treating a medical condition associated with a pathogenic microorganism in which an antimicrobial resistance has emerged against an antimicrobial agent in a subject having said medical condition as a result of treating said subject with said antimicrobial agent, the method comprising:
administering to said subject, following a treatment with said antimicrobial agent and said emergence of said antimicrobial resistance, a re-sensitizing effective amount of a polymer which comprises a plurality of positively charged amino acid residues and at least one ω-amino-fatty acid residue, wherein said ω-amino-fatty acid residue is being covalently linked to at least two amino acid residues in said plurality of positively charged amino acid residues via the N-alpha of one amino acid residue and via the C-alpha of the other amino acid residue in said at least two amino acid residues; and
administering to said subject a therapeutically effective amount of said antimicrobial agent.

2. The method of claim 1, wherein said re-sensitizing effective amount is lower than a therapeutically effective amount of said polymer with respect to said pathogenic microorganism.

3. The method of claim 1, wherein said antimicrobial agent is administered concomitant with or subsequent to administering said polymer.

4. The method of claim 1, wherein said at least one ω-amino-fatty acid is linked to each of said amino acid residues via a peptide bond.

5. The method of claim 1, wherein said positively charged amino acid residues are lysine residues.

6. The method of claim 1, wherein said ω-amino-fatty acid residue is selected from the group consisting of 4-amino-butyric acid residue, 8-amino-caprylic acid residue, 10-amino-decanoic acid residue, 12-amino-lauric acid residue, 14-amino-tetradecanoic acid residue and 16-amino-palmitic acid residue.

7. The method of claim 1, wherein said polymer comprises at least one fatty acid residue.

8. The method of claim 1, wherein said polymer has the general Formula I or II: 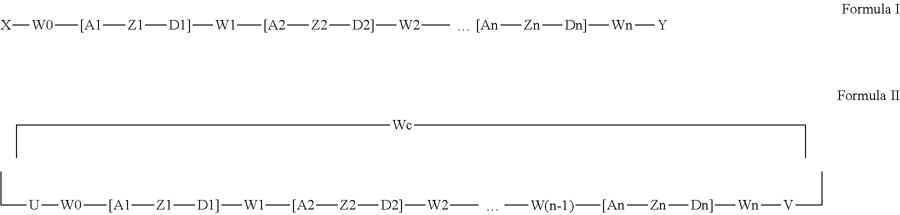
wherein:
n is an integer from 2 to 50;
A₁ to An are each independently a positively charge amino acid residue;
D₁ to Dn are each independently an ω-amino-fatty acid residue or absent, provided that at least one of said D₁ to Dn is said ω-amino-fatty acid residue;
Z₁ to Zn and W₀ and W₁ to Wn are each independently a linking moiety linking an amino acid residue and a hydrophobic moiety residue, or absent;
X and Y are each independently selected from the group consisting of hydrogen, amine, amide, a positively charged amino acid residue, an ω-amino-fatty acid residue, a fatty acid residue or absent;
W₀ is a linking moiety linking one of said A₁, Z₁ and D₁ to U, or absent;
Wn is a linking moiety linking one of said An, Zn and Dn to V, or absent;
U is selected from the group consisting of a first functional group, an amino acid residue having said first functional group, a hydrophobic moiety residue having said first functional group, and a linking moiety having said first functional group or absent;
V is selected from the group consisting of a second functional group, an amino acid residue having said second functional group, a hydrophobic moiety residue having said second functional group, and a linking moiety having said second functional group or absent; and
Wc is a cyclizing moiety.

9. The method of claim 1, wherein said re-sensitizing effective amount of said polymer is lower than 1 MIC unit.

10. The method of claim 1, wherein said re-sensitizing effective amount of said polymer ranges from ½ MIC units to ⅛ MIC unit.

11. The method of claim 1, wherein said polymer is selected from the group consisting of NC12(KNC12K)2NH2 (SEQ ID NO: 1), C12(5-ene)KKNC12KNH2 (SEQ ID NO: 2), C12K(NC8K)5NH2 (SEQ ID NO: 3), C12K(NC8K)7NH2 (SEQ ID NO: 4), C14(9-ene)KKNC12KNH2 (SEQ ID NO: 5), C16(9-ene)KKNC12KNH2 (SEQ ID NO: 6), C12KKNC12KNH2 (SEQ ID NO: 7), C12K(KNC12K)2NH2 (SEQ ID NO: 8), C12K(KNC12K)3NH2 (SEQ ID NO: 9) and C12K(KNC10K)3NH2 (SEQ ID NO: 10).

12. The method of claim 1, wherein said pathogenic microorganism is selected from the group consisting of Staphylococcus aureus, Pseudomonas aeruginosa, Proteus mirabilis, Stenotrophomonas maltophila, Bacillus cereus and Escherichia coli.

13. The method of claim 1, wherein said antimicrobial agent is selected from the group consisting of oxacillin, piperacillin, penicillin G, ciprofloxacin, erythromycin, tetracycline, gentamicin and methicillin.

14. A pharmaceutical composition comprising, as active ingredients, a polymer which comprises a plurality of positively charged amino acid residues and at least one ω-amino-fatty acid residue, wherein said ω-amino-fatty acid residue is being covalently linked to at least two amino acid residues in said plurality of positively charged amino acid residues via the N-alpha of one amino acid residue and via the C-alpha of the other amino acid residue in said at least two amino acid residues and an antimicrobial agent, and a pharmaceutically acceptable carrier,
the composition being packaged in a packaging material and identified in print, in or on said packaging material, for use in the treatment of a medical condition associated with a pathogenic microorganism in which an antimicrobial resistance has emerged against an antimicrobial agent in a subject having said medical condition as a result of treating said subject with said antimicrobial agent,
wherein said antimicrobial agent is other than said polymer, and said re-sensitizing effective amount is lower than a therapeutically effective amount of said polymer with respect to said pathogenic microorganism.

15. The pharmaceutical composition of claim 14, wherein said at least one ω-amino-fatty acid is linked to each of said amino acid residues via a peptide bond.

16. The pharmaceutical composition of claim 14, wherein said positively charged amino acid residues are lysine residues.

17. The pharmaceutical composition of claim 14, wherein said ω-amino-fatty acid residue is selected from the group consisting of 4-amino-butyric acid residue, 8-amino-caprylic acid residue, 10-amino-decanoic acid residue, 12-amino-lauric acid residue, 14-amino-tetradecanoic acid residue and 16-amino-palmitic acid residue.

18. The pharmaceutical composition of claim 14, wherein said polymer comprises at least one fatty acid residue.

19. The pharmaceutical composition of claim 14, wherein said polymer has the general Formula I or II: 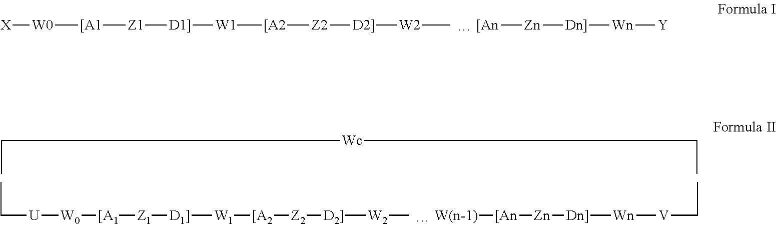
wherein:
n is an integer from 2 to 50;
A₁, A₂, . . . , An are each independently a positively charge amino acid residue;
D₁, D₂, . . . , Dn are each independently an ω-amino-fatty acid residue or absent, provided that at least one of said D₁, D₂, . . . , Dn is said ω-amino-fatty acid residue;
Z₁, Z₂, . . . , Zn and W₀, W₁, W₂, . . . , Wn are each independently a linking moiety linking an amino acid residue and a hydrophobic moiety residue, or absent;
X and Y are each independently selected from the group consisting of hydrogen, amine, amide, a positively charged amino acid residue, an ω-amino-fatty acid residue, a fatty acid residue or absent;
W₀ is a linking moiety linking one of said A₁, Z₁ and D₁ to U, or absent;
Wn is a linking moiety linking one of said An, Zn and Dn to V, or absent;
U is selected from the group consisting of a first functional group, an amino acid residue having said first functional group, a hydrophobic moiety residue having said first functional group, and a linking moiety having said first functional group or absent;
V is selected from the group consisting of a second functional group, an amino acid residue having said second functional group, a hydrophobic moiety residue having said second functional group, and a linking moiety having said second functional group or absent; and
Wc is a cyclizing moiety.

20. The pharmaceutical composition of claim 14, wherein said re-sensitizing effective amount of said polymer is lower than 1 MIC unit.

21. The pharmaceutical composition of claim 14, wherein said re-sensitizing effective amount of said polymer ranges from ½ MIC units to ⅛ MIC unit.

22. The pharmaceutical composition of claim 14, wherein said polymer is selected from the group consisting of NC12(KNC12K)2NH2 (SEQ ID NO: 1), C12(5-ene)KKNC12KNH2 (SEQ ID NO: 2), C12K(NC8K)5NH2 (SEQ ID NO: 3), C12K(NC8K)7NH2 (SEQ ID NO: 4), C14(9-ene)KKNC12KNH2 (SEQ ID NO: 5), C16(9-ene)KKNC12KNH2 (SEQ ID NO: 6), C12KKNC12KNH2 (SEQ ID NO: 7), C12K(KNC12K)2NH2 (SEQ ID NO: 8), C12K(KNC12K)3NH2 (SEQ ID NO: 9) and C12K(KNC10K)3NH2 (SEQ ID NO: 10).

23. The pharmaceutical composition of claim 14, wherein said antimicrobial agent is selected from the group consisting of oxacillin, piperacillin, penicillin G, ciprofloxacin, erythromycin, tetracycline, gentamicin and methicillin.


SEQUENCE LISTING (10 sequences)

SEQ ID NO: 1 - protein, 4 aa, Artificial sequence
  Synthetic peptide
  misc_feature: C′ amine
  misc_feature (1)..(1): Omega-amino-12 carbon fatty acid-lysine- conjugate residue
  misc_feature (2)..(2): Omega-amino-12 carbon fatty acid-lysine- conjugate residue
  misc_feature (4)..(4): Omega-amino-12 carbon fatty acid-lysine- conjugate residue
XXKX

SEQ ID NO: 2 - protein, 3 aa, Artificial sequence
  Synthetic peptide
  misc_feature: C′ amidated peptide
  misc_feature (1)..(1): N′ 12-carbon fatty acid (5-ene) residue conjugated lysine
  misc_feature (3)..(3): Omega-amino-12 carbon fatty acid-lysine- conjugate residue
KKX

SEQ ID NO: 3 - protein, 6 aa, Artificial sequence
  Synthetic peptide
  misc_feature: C′ amine
  misc_feature (1)..(1): N′ 12-carbon fatty acid residue conjugated lysine
  misc_feature (2)..(6): Omega-amino-8 carbon fatty acid-lysine- conjugate residue
KXXXXX

SEQ ID NO: 4 - protein, 8 aa, Artificial sequence
  Synthetic peptide
  misc_feature: C′ amine
  misc_feature (1)..(1): N′ 12-carbon fatty acid residue conjugated lysine
  misc_feature (2)..(8): Omega-amino-8 carbon fatty acid-lysine- conjugate residue
KXXXXXXX

SEQ ID NO: 5 - protein, 3 aa, Artificial sequence
  Synthetic peptide
  misc_feature: C′ amidated peptide
  misc_feature (1)..(1): N′ 14-carbon (9-ene) fatty acid residue conjugated lysine
  misc_feature (3)..(3): Omega-amino-12 carbon fatty acid-lysine- conjugate residue
KKX

SEQ ID NO: 6 - protein, 3 aa, Artificial sequence
  Synthetic peptide
  misc_feature: C′ amidated peptide
  misc_feature (1)..(1): N′ 16-carbon (9-ene) fatty acid residue conjugated lysine
  misc_feature (3)..(3): Omega-amino-12 carbon fatty acid-lysine- conjugate residue
KKX

SEQ ID NO: 7 - protein, 3 aa, Artificial sequence
  Synthetic peptide
  misc_feature: C′ amine
  misc_feature (1)..(1): N′ 12-carbon fatty acid residue conjugated lysine
  misc_feature (3)..(3): Omega-amino-12 carbon fatty acid-lysine- conjugate residue
KKX

SEQ ID NO: 8 - protein, 5 aa, Artificial sequence
  Synthetic peptide
  misc_feature: C′ amine
  misc_feature (1)..(1): N′12-carbon fatty acid residue conjugated lysine
  misc_feature (3)..(3): Omega-amino-12 carbon fatty acid-lysine- conjugate residue
  misc_feature (5)..(5): Omega-amino-12 carbon fatty acid-lysine- conjugate residue
KKXKX

SEQ ID NO: 9 - protein, 7 aa, Artificial sequence
  Synthetic peptide
  misc_feature: C′ amine
  misc_feature (1)..(1): N′12-carbon fatty acid residue conjugated lysine
  misc_feature (3)..(3): Omega-amino-12 carbon fatty acid-lysine- conjugate residue
  misc_feature (5)..(5): Omega-amino-12 carbon fatty acid-lysine- conjugate residue
  misc_feature (7)..(7): Omega-amino-12 carbon fatty acid-lysine- conjugate residue
KKXKXKX

SEQ ID NO: 10 - protein, 7 aa, Artificial sequence
  Synthetic peptide
  misc_feature: C′ amine
  misc_feature (1)..(1): N′12-carbon fatty acid residue conjugated lysine
  misc_feature (3)..(3): Omega-amino-10 carbon fatty acid-lysine- conjugate residue
  misc_feature (5)..(5): Omega-amino-10 carbon fatty acid-lysine- conjugate residue
  misc_feature (7)..(7): Omega-amino-10 carbon fatty acid-lysine- conjugate residue
KKXKXKX